(19) United States
(12) Patent Application Publication
(10) Pub. No.: US 2025/0339536 A1
(43) Pub. Date: Nov. 6, 2025

(54) BIOPHARMACEUTICAL FORMULATIONS INCLUDING POLYMER EXCIPIENTS

(71) Applicant: The Board of Trustees of the Leland Stanford Junior University, Palo Alto, CA (US)

(72) Inventors: Eric A. APPEL, Palo Alto, CA (US); Caitlin MAIKAWA, Boston, MA (US); Leslee NGUYEN, Stanford, CA (US)

(21) Appl. No.: 18/881,142

(22) PCT Filed: Jul. 17, 2023
(86) PCT No.: PCT/US23/27945

§ 371 Date: Jan. 3, 2025

Related U.S. Application Data
(60) Provisional application No. 63/389,708, filed on Jul. 15, 2022.
(60) Provisional application No. 63/522,786, filed on Jun. 23, 2023.

(51) Int. Cl. A61K 47/32; A61K 38/28

(52) U.S. Cl. CPC A61K 47/32; A61K 38/28

(57) ABSTRACT

The present disclosure provides compositions including a polyacrylamide-based copolymer, one or more preservatives, and insulin or an analog thereof. Also provided are methods of using the insulin compositions, including methods of administering the compositions to a human subject in need thereof.

### RELATED APPLICATIONS

This application claims the benefit of the priority of U.S. Provisional Applications No. 63/389,708, filed Jul. 15, 2023, and No. 63/522,786, filed Jun. 23, 2023, each of which is incorporated herein by reference in its entirety.

### GOVERNMENT RIGHTS

This invention was made with government support under Grants DK119254 and DK116074 awarded by the National Institutes of Health (NIH). The government has certain rights in the invention.

### BACKGROUND

Insulin is an essential protein drug for over 20 million patients worldwide with type 1 diabetes. While insulin formulations have advanced tremendously in the last century, there is a continued push to develop ultra-rapid insulin formulations that would better mimic endogenous insulin secretion and improve automated insulin delivery in “artificial pancreas” devices. Yet, current rapid-acting analogues still fall short of the ultra-rapid response of insulin secreted from a healthy pancreas. Administering a formulation of insulin monomers would result in the fastest absorption rate from the subcutaneous space, but the insulin monomer is highly unstable in formulation, which has thus far prevented commercial use of this strategy. Understanding the role of formulation excipients on insulin association state and stability is an important step towards commercializing an ultra-fast insulin formulation.

Excipients are often overlooked as the “inactive” ingredients in pharmaceutical formulations. Yet, in many drug formulations excipients play a critical role in drug viability by improving solubility, absorption, and stability of the active ingredient. Insulin is naturally stored as a zinc-stabilized hexamer in healthy beta cells, and this form has been replicated in most commercial insulin formulations to maintain insulin stability and shelf-life. However, while zinc-stabilized hexamers dissociate almost instantaneously when secreted into the blood, the hexamers dissociate much slower in the subcutaneous space due to lower dilution effects. As a result, exogenously delivered insulin can have delayed onset and a longer duration of action dependent on the dissociation of the insulin hexamers first into dimers and then into the active monomers that are absorbed into the blood.

There is a need for improved formulations of insulin or insulin analogs.

### SUMMARY

The present disclosure provides compositions including a polyacrylamide-based copolymer, one or more preservatives, and insulin or an analog thereof. The inventors have demonstrated that particular polyacrylamide-based copolymers can be used as stabilizing excipients in formulations of insulin or an analog thereof, without interacting directly with the insulin, or altering its pharmacokinetic properties. The results presented herein indicate that the polyacrylamide-based copolymers of this disclosure can confer a substantial stability benefit to compositions including insulin or an analog thereof, by precluding adsorption of the insulin to the interfaces of the composition, thereby preventing undesirable aggregation events and maintaining the binding activity of the insulin. Also provided are methods of using the insulin compositions, including methods of administering the compositions to a human subject in need thereof.

In one aspect, a composition of the present disclosure includes: a polyacrylamide-based copolymer including: a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof, a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), N-phenylacrylamide (PHE), and combinations thereof, a preservative; and insulin, or an analog thereof.

In another aspect, the present disclosure contemplates a method of treating an elevated glucose level in a subject in need thereof comprising administering to the subject a therapeutically effective amount of a polyacrylamide-based copolymer including: a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof; a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), N-phenylacrylamide (PHE), and combinations thereof, a preservative; and insulin, or an analog thereof, wherein the elevated glucose level is associated with insulin deficiency in the subject.

In still another aspect, a method of managing a blood glucose level in a subject in need thereof includes administering to the subject a therapeutically effective amount of polyacrylamide-based copolymer including: a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof, a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), N-phenylacrylamide (PHE), and combinations thereof, a preservative; and insulin, or an analog thereof.

### BRIEF DESCRIPTION OF THE DRAWINGS

FIG. 1, panels A-B, shows a set of schematics of insulin association states, preservatives, and pharmacokinetics. (Panel A) Schematic showing how preservatives can promote different association states. (Panel B) Schematic showing how insulin pharmacokinetics can be shifted by altering insulin association state.

FIG. 2 is a set of graphs showing insulin lispro association states for various lispro formulations with differing amounts of excipients.

FIG. 3 is a set of graphs showing regular human insulin (RHI) associate states for various RHI formulations with differing compositions of excipients.

FIG. 4, panels A-D, shows a set of graphs illustrating insulin lispro formulation stability. (Panel A) Change in transmittance traces and (Panel B) Time to aggregation of formulations with different antimicrobial preservatives: lispro—1 (meta-cresol), lispro—2 (meta-cresol+phenoxyethanol), lispro—3 (phenoxyethanol), lispro—4 (methylparaben+propylparaben). (Panel C) Change in transmittance traces and (Panel D) Time to aggregation of phenoxyethanol formulation (lispro—3) with different concentrations of MoNi_{23%} polymer excipient.

FIG. 5, panels A-D, shows a set of graphs illustrating RHI formulation stability. (Panel A) Change in transmittance traces and (Panel B) Time to aggregation of formulations with different antimicrobial preservatives: RHI—1 (meta-cresol), RHI—2 (meta-cresol+phenoxyethanol), RHI—3 (phenoxyethanol), RHI—4 (methylparaben+propylparaben). (Panel C) Change in transmittance traces and (Panel D) Time to aggregation of phenoxyethanol formulation (RHI—3) with different concentrations of MoNi_{23%} polymer excipient.

FIG. 6, panels A-B, show a pair of graphs illustrating stability of exemplary lispro-3 formulations having different glycerol concentrations. A change in glycerol concentration from 1.6 wt. % as is common in commercial formulations to 2.6 wt. % was shown not to have an effect on formulation stability. (Panel A) Change in transmittance traces, and (Panel B) Time to aggregation.

FIG. 7 is graph comparing stability of regular human insulin (RHI) formulations with or without a MoNi polymer excipient in a stressed aging assay (continuous agitation at 50° C.). Formulation A (dotted line): U400 RHI without MoNi; Formulation B (broken gray line): a commercial recombinant human insulin formation (INSUMAN® U400, Sanofi-Aventis); and Formulation C (solid black line): U400 RHI with 1 mg/mL MoNi. Formation of insulin aggregates in the stressed aging assay leads to light scattering and a reduction in the transmittance over time.

### DETAILED DESCRIPTION OF EMBODIMENTS

As summarized above, this disclosure relates to compositions including insulin or an analog thereof, and excipients including a preservative and a polyacrylamide-based copolymer. In some embodiments, the insulin or insulin analog is present in the composition substantially in a tetrameric- or lower-order association state. In some embodiments, the insulin or insulin analog is present in the composition substantially in a monomeric association state. In some embodiments, the composition has significantly better stability relative to commercial formulations. In some embodiments, the composition has a significantly shorter peak action relative to commercial formulations. In some embodiments, the composition has a significantly faster rate of absorption relative to commercial formulations.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### 1.1. Compositions

Provided herein are compositions including a polyacrylamide-based copolymer and insulin, or an analog thereof. The polyacrylamide-based copolymer contains a water-soluble carrier monomer and a functional dopant monomer. The subject compositions also include one or more preservatives. In some embodiments, the preservative is selected from meta-cresol, phenoxyethanol, methylparaben, propylparaben, and combinations thereof. In some embodiments, the compositions are aqueous. In some embodiments, the composition is substantially free from zinc. In some embodiments, the composition including less than 0.1 wt % of zinc, such as less than 0.05 wt %, less than 0.01 wt %, or less than 0.001 wt % of zinc in the composition. The incorporation of the polyacrylamide-based copolymer into the composition can prevent or reduce aggregation of the insulin, thereby maintaining the biological activity of the insulin.

As used herein, the term “association state,” used in reference to insulin or an insulin analog, describes the extent of aggregation of insulin or an analog thereof. For example, non-aggregated insulin present in a composition can be referred to as monomeric insulin, or insulin present in a monomeric association state. In another example, hexamers of insulin present in a composition can be referred to as hexameric insulin, or insulin present in a hexameric association state. When used in reference to one or more given association states, the terms “higher-order” and “lower-order” association state refers to association states greater than, or less than, those given association states, respectively.

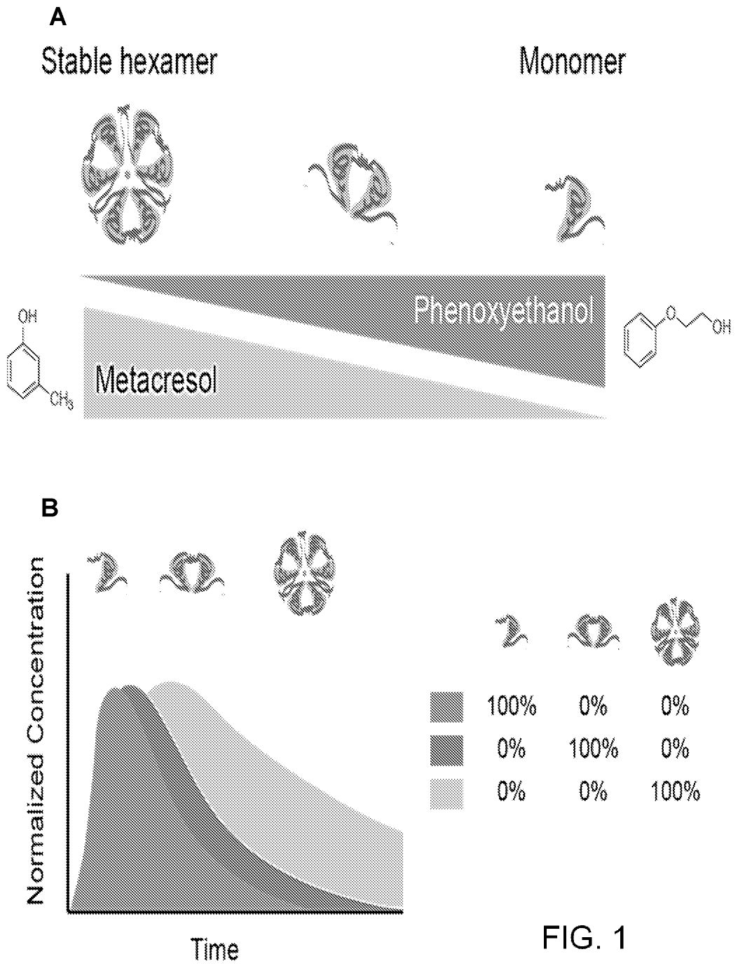

FIG. 1 shows two schematics of various insulin association states. The schematic in Panel A illustrates how preservatives can promote different association states. The schematic in Panel B illustrates how insulin pharmacokinetics can be shifted by altering insulin association state.

The term “substantially in a monomeric association state,” used in reference to an insulin composition, refers to compositions in which 50% or more of the insulin, or analog thereof, therein is present in a monomeric association state. In some embodiments, 60% or more, 70% or more, 80% or more, 90% or more, 95% or more, or 97% or more of the insulin, or analog thereof, that is present in an insulin composition of this disclosure is in a monomeric association state. The term “stability,” used in reference to an insulin composition, refers to the ability of the composition to retain at least a portion of binding activity of the insulin or analog thereof therein over time. For example, a “stable” composition can, in some embodiments, retain 70% or more (e.g., 80% or more, 90% or more, or 95% or more) of a binding activity of insulin or an analog thereof therein over 21 days or more of continuous stressed aging (e.g., based upon half-maximal inhibitory concentration as determined by an enzyme-linked immunosorbent assay).

In some embodiments, the composition is zinc-free. In some embodiments, the composition is substantially free from zinc. In some embodiments, the composition includes less than 0.1 wt % of zinc, such as less than 0.05 wt %, less than 0.01 wt %, or less than 0.001 wt % of zinc. It is understood that the term “zinc” refers to various salt forms or complexes of zinc including zinc ions, e.g., Zn²⁺ ions.

In some embodiments, the composition is substantially free from meta-cresol. In some embodiments, the composition substantially free from meta-cresol includes less than 0.5 wt % of meta-cresol. In some embodiments, the composition includes less than 0.25 wt % of meta-cresol, such as less than 0.1 wt % of meta-cresol, such as less than 0.05 wt %, less than 0.01 wt %, or less than 0.001 wt % of meta-cresol.

In some embodiments, the composition has a peak action of less than about 30 minutes, for example, less than about 20 minutes, less than about 15 minutes, less than about 12 minutes, or less than about 10 minutes. In some embodiments, the peak action is determined in a swine model of insulin deficient diabetes. In some embodiments, the composition has a duration of action of less than about 150 minutes, for example, less than about 120 minutes, less than about 90 minutes, less than about 60 minutes, less than about 40 minutes, or less than about 30 minutes. In some embodiments, the duration of action is determined in a rat model of insulin deficient diabetes.

### 1.1.1. Polyacrylamide-Based Copolymers

The term “polyacrylamide-based copolymer” refers to a polymer that is formed from the polymerization of two or more distinct monomers, in which at least one of the monomers possesses an acrylamide functional group (i.e., acrylamide monomer). In some embodiments, the polyacrylamide-based copolymer is formed from the polymerization of two structurally different acrylamide monomers (two structurally different monomers that each possess an acrylamide functional group).

The resulting copolymer can be an alternating copolymer wherein the monomer species are connected in an alternating fashion; a random copolymer, wherein the monomer species are connected to each other within a polymer chain without a defined pattern; a block copolymer, wherein polymeric blocks of one monomer species are connected to polymeric blocks made up of another monomer species; and graft copolymer, wherein the main polymer chain consists of one monomer species, and polymeric blocks of another monomer species are connected to the main polymer chain as side branches (also referred to as sidechains). In some embodiments, the polyacrylamide-based copolymers of the present disclosure are random copolymers.

An “acrylamide monomer,” refers to a monomer species that possesses an acrylamide functional group. The term “acrylamide monomer” includes not only monomeric acrylamide, but derivatives of monomeric acrylamide. Examples of acrylamide monomers include, but are not limited to, acrylamide (AM), N-(3-methoxypropoyl)acrylamide (MPAM), 4-acryloylmorpholine (MORPH), N,N-dimethylacrylamide (DMA), N-hydroxyethyl acrylamide (HEAM), N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), and N-phenylacrylamide (PHE).

In some embodiments, the polyacrylamide-based copolymer of the compositions of the present disclosure is amphiphilic. In some embodiments, the polyacrylamide-based copolymers are co-polymers of two acrylamide monomers, a water-soluble carrier monomer and a functional dopant monomer. In some embodiments, the polyacrylamide-based copolymer is formed via a random polymerization of a water-soluble carrier monomer and a functional dopant monomer.

As defined herein, the term “water-soluble carrier monomer” refers to an acrylamide monomer species that is the water-soluble monomer species within the polyacrylamide-based copolymer. In some embodiments, the water-soluble carrier monomer is the predominant hydrophilic species within the polyacrylamide-based copolymer. In some embodiments, the water-soluble carrier monomer provides a hydrophilic sidechain group that imparts aqueous solubility to the copolymer.

In some embodiments, the water-soluble carrier monomer within the polyacrylamide-based copolymer provides an inert barrier at an interface of an aqueous formulation to prevent protein-protein interactions. In some embodiments, the interface is an air-water interface. In some embodiments, the interface is an enclosure-water interface, including, but not limited to, a glass-water interface, a rubber-water interface, a plastic-water interface, or a metal-water interface. In some embodiments, the interface is an oil-water interface. In some embodiments, the interface is an interface between a liquid and tubing. In some embodiments, the interface is an interface between a liquid and a catheter. In some embodiments, the enclosure-water interface is in a pump system. In some embodiments, the enclosure-water interface is in a closed-loop system.

In some embodiments, the water-soluble carrier monomer is hydrophilic and/or nonionic. Examples of water-soluble carrier monomers of interest include, but are not limited to, acrylamide (AM), N-(3-methoxypropoyl)acrylamide (MPAM), 4-acryloylmorpholine (MORPH), N,N-dimethylacrylamide (DMA), and N-hydroxyethyl acrylamide (HEAM).

The term “functional dopant monomer,” as used herein, refers to an acrylamide monomer species that has one or more physicochemical properties (e.g., hydrophobicity, charge, etc.) different from those of the water-soluble carrier monomer. In some embodiments, the functional dopant monomer within the polyacrylamide-based copolymer promotes association of the polymers to an interface of the composition; such interfaces can include, but are not limited to, polymer-air-water interface interactions, polymer-protein interactions, polymer-peptide interactions, polymer-micelle interactions, polymer-liposome interactions, and polymer-lipid nanoparticle interactions. The functional dopant monomer can act as a stabilizing moiety to facilitate interactions with biomolecules, for example, proteins, peptides, antibodies, antibody-drug conjugates, nucleic acids, lipid particles, and combinations thereof (e.g., to prevent aggregation of the biomolecules). The functional dopant monomers can be further classified into hydrogen-bonding, ionic, hydrophobic, and aromatic monomers based on their chemical composition. Typically, the functional dopant monomers are copolymerized at a lower weight percentage in the co-polymer as compared to the water-soluble carrier monomers.

The term “polymerization” refers to the process in which monomer molecules undergo a chemical reaction to form polymeric chains or three-dimensional networks. Different types of polymerization reactions are known in the art, for example, addition (chain-reaction) polymerization, condensation polymerization, ring-opening polymerization, free radical polymerization, controlled radical polymerization, atom transfer radical polymerization (ATRP), single-electron transfer living radical polymerization (SET-LRP), reversible addition-fragmentation chain transfer (RAFT) polymerization, nitroxide-mediated polymerization (NMP), and emulsion polymerization. The polymerization reaction can be a vinyl addition polymerization initiated via a free radical generating system. In some embodiments, the copolymers of the present disclosure are prepared using RAFT polymerization.

The term “degree of polymerization” (DP) refers to the number of monomer units in a polymer. It is calculated by dividing the average molecular weight of a polymer sample by the molecular weight of the monomers. The average molecular weight of a polymer can be represented by the number-averaged molecular weight (Mn), the weight-average molecular weight (Mw), the Z-average molecular weight (Mz) or the molecular weight at the peak maxima of the molecular weight distribution curve (Mp). The average molecular weight of a polymer can be determined by a variety of analytical characterization techniques known to those skilled in the art, for example, gel permeation chromatography (GPC), static light scattering (SLS) analysis, multi-angle laser light scattering (MALLS) analysis, nuclear magnetic resonance spectroscopy (NMR), intrinsic viscometry (IV), melt flow index (MFI), and matrix-assisted laser desorption/ionization mass spectrometry (MALDI-MS), and combinations thereof. Degree of polymerization can also be determined experimentally using suitable analytical methods known in the art, such as nuclear magnetic spectroscopy (NMR), Fourier Transform infrared spectroscopy (FT-IR) and Raman spectroscopy.

The compositions described herein can include a polyacrylamide-based copolymer comprising: a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof, and a functional dopant monomer selected from N-[tris(hydroxymethyl)-ethyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), N-phenylacrylamide (PHE), and combinations thereof.

In some embodiments, the water-soluble carrier monomer of the polyacrylamide-based copolymer is selected from MORPH, MPAM, and combinations thereof. In some embodiments, the water-soluble carrier monomer includes MORPH or MPAM. In some embodiments, the water-soluble carrier monomer is MORPH. In some embodiments, the water-soluble carrier monomer is MPAM.

In some embodiments, the functional dopant monomer of the polyacrylamide-based copolymer is selected from AMP, TMA, TBA, PHE, and combinations thereof. In some embodiments, the functional dopant monomer includes TRI, PHE, or NIP. In some embodiments, the functional dopant monomer includes DEA, PHE, or NIP. In some embodiments, the functional dopant monomer is NIP. In some embodiments, the functional dopant monomer is PHE. In some embodiments, the functional dopant monomer is DEA.

In some embodiments, the water-soluble carrier monomer is selected from MPAM, MORPH, and combinations thereof, and the functional dopant monomer is selected from NIP, PHE, and combinations thereof. In some embodiments, the water-soluble carrier monomer is selected from MPAM, MORPH, and combinations thereof, and the functional dopant monomer is selected from DEA, NIP, PHE, and combinations thereof. In some embodiments, the water-soluble carrier monomer is selected from MPAM, MORPH, and combinations thereof, and the functional dopant monomer is selected from AMP, TMA, TBA, PHE, and combinations thereof.

In some embodiments, the water-soluble carrier monomer includes MPAM, and the functional dopant monomer includes PHE.

In some embodiments, the water-soluble carrier monomer includes MPAM, and the functional dopant monomer includes NIP.

In some embodiments, the water-soluble carrier monomer includes MORPH, and the functional dopant monomer includes PHE.

In some embodiments, the water-soluble carrier monomer includes MORPH, and the functional dopant monomer includes NIP. Polyacrylamide-based copolymers wherein the water-soluble carrier monomer includes MORPH, and the functional dopant monomer includes NIP are also described herein as “MoNi”.

In some embodiments, the water-soluble carrier monomer includes MORPH, and the functional dopant monomer includes DEA.

In some embodiments, the water-soluble carrier monomer includes MPAM, and the functional dopant monomer includes DEA.

In some embodiments, the water-soluble carrier monomer includes MORPH, and the functional dopant monomer includes AMP. In some embodiments, the water-soluble carrier monomer includes MORPH, and the functional dopant monomer includes TMA. In some embodiments, the water-soluble carrier monomer includes MORPH, and the functional dopant monomer includes TBA. In some embodiments, the water-soluble carrier monomer includes MORPH, and the functional dopant monomer includes TRI.

In some embodiments, the water-soluble carrier monomer includes MPAM, and the functional dopant monomer includes AMP. In some embodiments, the water-soluble carrier monomer includes MPAM, and the functional dopant monomer includes TMA. In some embodiments, the water-soluble carrier monomer includes MPAM, and the functional dopant monomer includes TBA. In some embodiments, the water-soluble carrier monomer includes MPAM, and the functional dopant monomer includes TRI.

In some embodiments, the copolymer contains about 70 wt % to about 98 wt % of the water-soluble carrier monomer, for example, about 70 wt % to about 95 wt %, about 70 wt % to about 90 wt %, about 75 wt % to about 98 wt %, about 75 wt % to about 95 wt %, about 75 wt % to about 90 wt %, about 80 wt % to about 98 wt %, about 80 wt % to about 95 wt %, about 80 wt % to about 90 wt %, about 83 wt % to about 98 wt %, about 83 wt % to about 95 wt %, or about 83 wt % to about 90 wt % of the water-soluble carrier monomer. In some embodiments, the copolymer contains about 2 wt % to about 30 wt % of the functional dopant monomer, for example, about 2 wt % to about 20 wt %, about 2 wt % to about 17 wt %, about 5 wt % to about 30 wt %, about 5 wt % to about 20 wt %, about 5 wt % to about 17 wt %, about 10 wt % to about 30 wt %, about 10 wt % to about 20 wt %, or about 10 wt % to about 17 wt % of the functional dopant monomer.

In some embodiments, the copolymer contains 70 wt % to 98 wt % of the water-soluble carrier monomer, for example, 70 wt % to 95 wt %, 70 wt % to 90 wt %, 75 wt % to 98 wt %, 75 wt % to 95 wt %, 75 wt % to 90 wt %, 80 wt % to 98 wt %, 80 wt % to 95 wt %, 80 wt % to 90 wt %, 83 wt % to 98 wt %, 83 wt % to 95 wt %, or 83 wt % to 90 wt % of the water-soluble carrier monomer. In some embodiments, the copolymer contains 2 wt % to 30 wt % of the functional dopant monomer, for example, 2 wt % to 20 wt %, 2 wt % to 17 wt %, 5 wt % to 30 wt %, 5 wt % to 20 wt %, 5 wt % to 17 wt %, 10 wt % to 30 wt %, 10 wt % to 20 wt %, or 10 wt % to 17 wt % of the functional dopant monomer.

In some embodiments, the copolymer contains about 70 wt % to about 85 wt %, about 70 wt % to about 80 wt %, about 74 wt % to about 85 wt %, about 74 wt % to about 80 wt %, or about 77 wt % of MORPH. In some embodiments, the copolymer contains about 15 wt % to about 30 wt %, about 15 wt % to about 26 wt %, about 20 wt % to about 30 wt %, about 20 wt % to about 26 wt %, or about 23 wt % of NIP. In some embodiments, the copolymer contains 70 wt % to 85 wt %, 70 wt % to 80 wt %, 74 wt % to 85 wt %, 74 wt % to 80 wt %, or 77 wt % of MORPH. In some embodiments, the copolymer contains 15 wt % to 30 wt %, 15 wt % to 26 wt %, 20 wt % to 30 wt %, 20 wt % to 26 wt %, or 23 wt % of NIP.

In some embodiments, the copolymer contains about 80 wt % to about 99 wt %, about 85 wt % to about 98 wt %, about 88 wt % to about 96 wt %, about 90 wt % to about 95 wt %, or about 94 wt % of MORPH. In some embodiments, the copolymer contains about 2 wt % to about 15 wt %, about 4 wt % to about 15 wt %, about 4 wt % to about 10 wt %, about 5 wt % to about 10 wt %, or about 6 wt % of PHE. In some embodiments, the copolymer contains 80 wt % to 99 wt %, 85 wt % to 98 wt %, 88 wt % to 96 wt %, 90 wt % to 95 wt %, or 94 wt % of MORPH. In some embodiments, the copolymer contains 2 wt % to 15 wt %, 4 wt % to 15 wt %, 4 wt % to 10 wt %, 5 wt % to 10 wt %, or 6 wt % of PHE.

In some embodiments, the copolymer contains about 80 wt % to about 99 wt %, about 85 wt % to about 98 wt %, about 88 wt % to about 96 wt %, about 90 wt % to about 95 wt %, or about 92 wt % of MORPH. In some embodiments, the copolymer contains about 2 wt % to about 15 wt %, about 4 wt % to about 15 wt %, about 4 wt % to about 10 wt %, about 5 wt % to about 10 wt %, or about 8 wt % of PHE. In some embodiments, the copolymer contains 80 wt % to 99 wt %, 85 wt % to 98 wt %, 88 wt % to 96 wt %, 90 wt % to 95 wt %, or 92 wt % of MORPH. In some embodiments, the copolymer contains 2 wt % to 15 wt %, 4 wt % to 15 wt %, 4 wt % to 10 wt %, 5 wt % to 10 wt %, or 8 wt % of PHE.

In some embodiments, a degree of polymerization of the copolymer is about 10 to about 500, for example, about 10 to about 350, about 10 to about 200, about 15 to about 500, about 15 to about 350, about 15 to about 200, about 20 to about 500, about 20 to about 350, or about 20 to about 200. In some embodiments, a degree of polymerization of the copolymer is 10 to 500, for example, 10 to 350, 10 to 200, 15 to 500, 15 to 350, 15 to 200, 20 to 500, 20 to 350, or 20 to 200.

In some embodiments, a number-average molecular weight of the copolymer is about 2,000 g/mol to about 10,000 g/mol, for example, about 2,000 g/mol to about 7,500 g/mol, 2,000 g/mol to about 6,000 g/mol, about 2,000 g/mol to about 5,000 g/mol, about 3,000 g/mol to about 10,000 g/mol, about 3,000 g/mol to about 7,500 g/mol, about 3,000 g/mol to about 6,000 g/mol, or about 3,000 g/mol to about 5,000 g/mol. In some embodiments, a number-average molecular weight of the copolymer is 2,000 g/mol to 10,000 g/mol, for example, 2,000 g/mol to 7,500 g/mol, 2,000 g/mol to 6,000 g/mol, 2,000 g/mol to 5,000 g/mol, 3,000 g/mol to 10,000 g/mol, 3,000 g/mol to 7,500 g/mol, 3,000 g/mol to 6,000 g/mol, or 3,000 g/mol to 5,000 g/mol.

In some embodiments, the composition includes about 0.001 wt % to about 1 wt % of the copolymer, for example, about 0.001 wt % to about 0.5 wt %, about 0.001 wt % to about 0.1 wt %, about 0.001 wt % to about 0.05 wt %, about 0.001 wt % to about 0.03 wt %, about 0.005 wt % to about 1 wt %, about 0.005 wt % to about 0.5 wt %, about 0.005 wt % to about 0.1 wt %, about 0.005 wt % to about 0.05 wt %, about 0.005 wt % to about 0.03 wt %, about 0.01 wt % to about 1 wt %, about 0.01 wt % to about 0.5 wt %, about 0.01 wt % to about 0.1 wt %, about 0.01 wt % to about 0.05 wt %, or about 0.01 wt % to about 0.03 wt % of the copolymer. In some embodiments, the composition includes about 0.01 wt % of the copolymer. In some embodiments, the composition includes 0.001 wt % to 1 wt % of the copolymer, for example, 0.001 wt % to 0.5 wt %, 0.001 wt % to 0.1 wt %, 0.001 wt % to 0.05 wt %, 0.001 wt % to 0.03 wt %, 0.005 wt % to 1 wt %, 0.005 wt % to 0.5 wt %, 0.005 wt % to 0.1 wt %, 0.005 wt % to 0.05 wt %, 0.005 wt % to 0.03 wt %, 0.01 wt % to 1 wt %, 0.01 wt % to 0.5 wt %, 0.01 wt % to 0.1 wt %, 0.01 wt % to 0.05 wt %, or 0.01 wt % to 0.03 wt % of the copolymer. In some embodiments, the composition includes 0.01 wt % of the copolymer.

### 1.1.2. Preservatives and Tonicity Modifiers

As summarized above, the insulin composition including the acrylamide-based copolymer (e.g., as described herein) includes one or more preservatives. In some embodiments, the subject compositions include a preservative selected from meta-cresol, phenoxyethanol, methylparaben, propylparaben, and combinations thereof. In some embodiments, the composition includes about 0.01 wt % to about 2 wt % of the preservative, for example, about 0.01 wt % to about 1.5 wt %, about 0.01 wt % to about 1 wt %, about 0.05 wt % to about 2 wt %, about 0.05 wt % to about 1.5 wt %, about 0.05 wt % to about 1 wt %, about 0.1 wt % to about 2 wt %, about 0.1 wt % to about 1.5 wt %, about 0.1 wt % to about 1 wt %, about 0.25 wt % to about 2 wt %, about 0.25 wt % to about 1.5 wt %, or about 0.25 wt % to about 1 wt % of the preservative. In some embodiments, the composition includes about 0.15 wt %, about 0.7 wt %, or about 0.85 wt % of the preservative. In some embodiments, the composition includes 0.01 wt % to 2 wt % of the preservative, for example, 0.01 wt % to 1.5 wt %, 0.01 wt % to 1 wt %, 0.05 wt % to 2 wt %, 0.05 wt % to 1.5 wt %, 0.05 wt % to 1 wt %, 0.1 wt % to 2 wt %, 0.1 wt % to 1.5 wt %, 0.1 wt % to 1 wt %, 0.25 wt % to 2 wt %, 0.25 wt % to 1.5 wt %, or 0.25 wt % to 1 wt % of the preservative. In some embodiments, the composition includes 0.15 wt %, 0.7 wt %, or 0.85 wt % of the preservative.

In some embodiments, the preservative includes meta-cresol. For example, in some embodiments, the composition includes about 0.01 wt % to about 1 wt %, about 0.01 wt % to about 0.75 wt %, about 0.01 wt % to about 0.5 wt %, about 0.01 wt % to about 0.25 wt %, about 0.01 wt % to about 0.1 wt %, about 0.1 wt % to about 1 wt %, about 0.1 wt % to about 0.75 wt %, or about 0.1 wt % to about 0.5 wt % of meta-cresol. In some embodiments, the composition includes about 0.32 wt % of meta-cresol. In some embodiments, the composition includes 0.01 wt % to 1 wt %, 0.01 wt % to 0.75 wt %, 0.01 wt % to 0.5 wt %, 0.01 wt % to 0.25 wt %, 0.01 wt % to 0.1 wt %, 0.1 wt % to 1 wt %, 0.1 wt % to 0.75 wt %, or 0.1 wt % to 0.5 wt % of meta-cresol. In some embodiments, the composition includes 0.32 wt % of meta-cresol.

In some embodiments, the preservative includes phenoxyethanol. For example, in some embodiments, the composition includes about 0.2 wt % to about 1.5 wt %, about 0.2 wt % to about 1.1 wt %, about 0.2 wt % to about 1 wt %, about 0.5 wt % to about 1.5 wt %, about 0.5 wt % to about 1.1 wt %, about 0.5 wt % to about 1 wt %, about 0.6 wt % to about 1.5 wt %, about 0.6 wt % to about 1.1 wt %, or about 0.6 wt % to about 1 wt % of phenoxy ethanol.

In some embodiments, the composition includes about 0.85 wt % of phenoxyethanol. In some embodiments, the composition includes 0.2 wt % to 1.5 wt %, 0.2 wt % to 1.1 wt %, 0.2 wt % to 1 wt %, 0.5 wt % to 1.5 wt %, 0.5 wt % to 1.1 wt %, 0.5 wt % to 1 wt %, 0.6 wt % to 1.5 wt %, 0.6 wt % to 1.1 wt %, or 0.6 wt % to 1 wt % of phenoxy ethanol. In some embodiments, the composition includes 0.85 wt % of phenoxyethanol.

In some embodiments, the preservative includes phenoxyethanol and meta-cresol. For example, in some embodiments, the composition includes about 0.01 wt % to about 1 wt %, or about 0.1 wt % to about 0.5 wt % of meta-cresol, and about 0.2 wt % to about 1.5 wt %, or about 0.6 wt % to about 1 wt % of phenoxy ethanol. In some embodiments, the composition includes 0.01 wt % to 1 wt %, or 0.1 wt % to 0.5 wt % of meta-cresol, and 0.2 wt % to 1.5 wt %, or 0.6 wt % to 1 wt % of phenoxy ethanol.

In some embodiments, the composition includes a preservative that is methylparaben, propylparaben, or both. In some embodiments, the composition includes about 0.05 wt % to about 0.3 wt %, about 0.05 wt % to about 0.25 wt %, about 0.05 wt % to about 0.2 wt %, about 0.07 wt % to about 0.3 wt %, about 0.07 wt % to about 0.25 wt %, about 0.07 wt % to about 0.2 wt %, about 0.09 wt % to about 0.3 wt %, about 0.09 wt % to about 0.25 wt %, or about 0.09 wt % to about 0.2 wt % of methylparaben. In some embodiments, the composition includes 0.05 wt % to 0.3 wt %, 0.05 wt % to 0.25 wt %, 0.05 wt % to 0.2 wt %, 0.07 wt % to 0.3 wt %, 0.07 wt % to 0.25 wt %, 0.07 wt % to 0.2 wt %, 0.09 wt % to 0.3 wt %, 0.09 wt % to 0.25 wt %, or 0.09 wt % to 0.2 wt % of methylparaben. In some embodiments, the composition includes about 0.001 wt % to about 0.1 wt %, about 0.001 wt % to about 0.05 wt %, about 0.001 wt % to about 0.03 wt %, about 0.005 wt % to about 0.1 wt %, about 0.005 wt % to about 0.05 wt %, about 0.005 wt % to about 0.03 wt %, about 0.01 wt % to about 0.1 wt %, about 0.01 wt % to about 0.05 wt %, or about 0.01 wt % to about 0.03 wt % of propylparaben. In some embodiments, the composition includes 0.001 wt % to 0.1 wt %, 0.001 wt % to 0.05 wt %, 0.001 wt % to 0.03 wt %, 0.005 wt % to 0.1 wt %, 0.005 wt % to 0.05 wt %, 0.005 wt % to 0.03 wt %, 0.01 wt % to 0.1 wt %, 0.01 wt % to 0.05 wt %, or 0.01 wt % to 0.03 wt % of propylparaben. In some embodiments, the composition includes 0.137 wt % of methylparaben, and 0.015 wt % of propylparaben.

In some embodiments, the composition includes a preservative that is the combination of propylparaben and methylparaben, for example, in a ratio (e.g., by ratios of wt %) of 1:10, 1:9, 1:8, 1:7, 1:6, 1:5, 1:4, 1:3, 1:2, or 1:1. In some embodiments, the composition includes propylparaben and methylparaben in a ratio of 1:10, 1:9.9, 1:9.8, 1:9.7, 1:9.6, 1:9.5, 1:9.4, 1:9:3, 1:9.2, 1:9:1, or 1:9. In some embodiments, the composition includes propylparaben and methylparaben in a ratio of 1:8 to 1:10, such as 1:9.

In some embodiments, the compositions described herein further include a tonicity modifier. In some embodiments, the composition includes about 0.5 wt % to about 5 wt %, for example, about 0.5 wt % to about 4 wt %, about 0.5 wt % to about 3 wt %, about 1 wt % to about 4 wt %, about 1 wt % to about 3 wt %, about 1.5 wt % to about 5 wt %, about 1.5 wt % to about 4 wt %, about 1.5 wt % to about 3 wt %, or about 2.6 wt % of a tonicity modifier. In some embodiments, the composition includes 0.5 wt % to 5 wt %, for example, 0.5 wt % to 4 wt %, 0.5 wt % to 3 wt %, 1 wt % to 4 wt %, 1 wt % to 3 wt %, 1.5 wt % to 5 wt %, 1.5 wt % to 4 wt %, 1.5 wt % to 3 wt %, or 2.6 wt % of a tonicity modifier. In some embodiments, the tonicity modifier includes sodium chloride, potassium chloride, mannitol, dextrose, glycerol (also known as glycerin), magnesium chloride, or a combination thereof. In some embodiments, the tonicity modifier includes sodium chloride, glycerol, or both. In some embodiments, the tonicity modifier includes glycerol.

In certain embodiments, the compositions described herein include a preservative (e.g., as described herein) and a tonicity modifier (such as glycerol), wherein (a) the composition includes 0.01 wt % to 2 wt % of the preservative, for example, 0.01 wt % to 1.5 wt %, 0.01 wt % to 1 wt %, 0.05 wt % to 2 wt %, 0.05 wt % to 1.5 wt %, 0.05 wt % to 1 wt %, 0.1 wt % to 2 wt %, 0.1 wt % to 1.5 wt %, 0.1 wt % to 1 wt %, 0.25 wt % to 2 wt %, 0.25 wt % to 1.5 wt %, or 0.25 wt % to 1 wt % of the preservative, such as meta-cresol, phenoxyethanol, methylparaben, propylparaben, and combinations thereof, and (b) the composition includes 0.5 wt % to 5 wt %, for example, 0.5 wt % to 4 wt %, 0.5 wt % to 3 wt %, 1 wt % to 4 wt %, 1 wt % to 3 wt %, 1.5 wt % to 5 wt %, 1.5 wt % to 4 wt %, 1.5 wt % to 3 wt %, or 2.6 wt % of a tonicity modifier, such as glycerol.

In some embodiments, the composition includes a tonicity modifier (such as glycerol) and a preservative (e.g., as described herein) in a ratio (by wt %) of 2:1 to 20:1, such as from 3:1 to 10:1, from 3:1 to 6:1, e.g., a ratio of about 3:1 or 4:1. In some embodiments, meta-cresol is absent from the composition.

In some embodiments, the composition includes copolymer and a preservative (e.g., as described herein) in a ratio (by wt %) of 1:10 to 1:100, such as from 1:50 to 1:100. In some embodiments, the preservative is phenoxyethanol. In some embodiments, meta-cresol is absent from the composition. In some embodiments, the composition includes copolymer and a preservative (e.g., as described herein) in a ratio (by wt %) of 1:10 to 1:50, such as from 1:10 to 1:20. In some embodiments, the preservative includes propylparaben and methylparaben e.g., as described herein). In some embodiments, meta-cresol is absent from the composition.

In some embodiments, the composition includes copolymer and a tonicity modifier (e.g., as described herein) in a ratio (by wt %) of 1:100 to 1:1000, such as from 1:100 to 1:500, from 1:150 to 1:300, or from 1:160 to 1:260. In some embodiments, the tonicity modifier is glycerol. In some embodiments, the copolymer is MoNi, such as MoNi_{23%}.

In some embodiments, the composition includes a copolymer, tonicity modifier (such as glycerol), and a preservative (e.g., as described herein, such as meta-cresol), wherein (a) the tonicity modifier and the preservative are in a ratio (by wt %) of 2:1 to 20:1, such as from 3:1 to 10:1, from 3:1 to 6:1, e.g., a ratio of about 3:1 or 4:1; and optionally(b) the copolymer and the preservative are in a ratio (by wt %) of 1:10 to 1:100, such as from 1:50 to 1:100; and optionally (c) the copolymer and the tonicity modifier are in a ratio (by wt %) of 1:100 to 1:1000, such as from 1:100 to 1:500, from 1:150 to 1:300, or from 1:160 to 1:260. In these embodiments, the preservative can be a combination of propylparaben and methylparaben, for example, in a ratio (e.g., by ratios of wt %) of 1:10, 1:9, 1:8, 1:7, 1:6, 1:5, 1:4, 1:3, 1:2, or 1:1, such as in a ratio of 1:10, 1:9.9, 1:9.8, 1:9.7, 1:9.6, 1:9.5, 1:9.4, 1:9:3, 1:9.2, 1:9:1, or 1:9, including in a ratio of 1:8 to 1:10, such as 1:9. In these embodiments, the tonicity modifier can be glycerol. In these embodiments, the copolymer can be MoNi, such as MoNi_{23%}.

In some embodiments, the composition includes a copolymer, tonicity modifier (such as glycerol), a preservative (e.g., as described herein, such as meta-cresol), wherein the copolymer:tonicity modifier:preservative are in a ratio (by wt %) of about 1:16:2.5.

In some embodiments the copolymer and the preservative are in a ratio (by wt %) of 1:10 to 1:1; such as 1:10 to 1:1.5, 1:10 to 1:2, 1:5 to 1:1, 1:5 to 1:1.5, or 1:5 to 1:2. In some embodiments, the copolymer and the preservative are in a ratio (by wt %) of 1:10, 1:9.5, 1:9, 1.8.5, 1:8, 1:7.5; 1:7, 1:6.5, 1:6, 1:5.5, 1:5, 1:4.5, 1:4, 1:3.5, 1:3, 1:2.5, 1:2, or 1:1 or any ranges created by using any of these ratios as endpoints, such as 1:4 to 1:1, 1.35 to 1.15, etc. In some embodiments, the copolymer can be MoNi, such as MoNi_{23%}. In some of such embodiments, the preservative is meta-cresol.

In some embodiments, the tonicity modifier and the preservative are in a ratio (by wt %) of 10:1 to 2:1, such as from 10:1 to 3:1 or from 10:1 to 5:1, e.g., a ratio of about 6:1 or 7:1. In some embodiments, the tonicity modifier and the preservative are in a ratio (by wt %) of 10:1, 9.5:1, 9:1, 8.5:1, 8:1, 7.5:1, 7:1, 6.5:1, 6.4:1, 6.3:1, 6:1, 5.5:1, 5:1, 4.5:1, 4:1, 3.5:1, 3:1, 2.5:1, or 2:1 or any ranges created by using any of these ratios as endpoints. In some embodiments, the tonicity modifier and the preservative are in a ratio (by wt %) of 6:1, 6.1:1, 6.2:1, 6.3:1, 6.4:1, 6.5:1, 6.6:1, 6.7:1, 6.8:1, 6.9:1, or 7:1 or any ranges created by using any of these ratios as endpoints, such as 6:1 to 7:1, 6.3:1 to 6.5:1, etc. In these embodiments, the tonicity modifier can be glycerol. In some of such embodiments, the preservative is meta-cresol. In these embodiments, the copolymer can be MoNi, such as MoNi_{23%}.

In some embodiments, the copolymer and the tonicity modifier are in a ratio (by wt %) of 1:30 to 1:5, such as from 1:25 to 1:7, from 1:19 to 1:13, or from 1:17 to 1:15, e.g., a ratio of about 1:16. In some embodiments, the copolymer and the tonicity modifier are in a ratio (by wt %) of 1:30, 1:28, 1:26, 1:24, 1:22, 1:20, 1:18, 1:16, 1:14, 1:12, 1:10, 1:8, 1:6, or 1:5 or any ranges created by using any of these ratios as endpoints, such as 1:26 to 1:6, 1:20 to 1:12, 1:18 to 1:14, etc. In these embodiments, the tonicity modifier can be glycerol. In these embodiments, the copolymer can be MoNi, such as MoNi_{23%}.

### 1.1.3. Insulin and Insulin Analogs

As summarized above, the disclosure provides compositions of insulin or an insulin analog formulated with a polyacrylamide-based copolymer. The inventors have demonstrated that polyacrylamide-based copolymers can be used to provide stable insulin formulations, such as an aqueous insulin formulation, without altering the pharmacokinetics or bioavailability. This formulation can be applied to a variety of insulin analogs.

The term “insulin” refers to a hormone produced by the beta cells in the pancreatic islets that regulates the amount of glucose in the blood. Many eukaryotes, including humans, primates, pigs, cows, cats, dogs, and rodents, produce insulin. Thus, “insulin,” as used herein, includes insulin produced by humans, and analogs thereof, as well as insulin, and analogs thereof, produced by other eukaryotes, including, but not limited to, primates, pigs, cows, cats, dogs, and rodents, and also includes recombinant, purified or synthetic insulin or insulin analogs having similar function and structure, unless otherwise specified. The human insulin protein consists of 51 amino acids and has a molecular weight of approximately 5.8 kilodalton (kDa). Human insulin is a heterodimer of an A-chain and a B-chain that are connected by disulfide bonds.

Insulin can be isolated from the pancreatic islets extracts of an animal that produces insulin, or expressed recombinantly in a suitable expression system such as E. coli, yeast, insect cells, and mammalian cells (e.g., Chinese hamster ovary (CHO) cells). Depending upon their specific pharmacokinetics and pharmacodynamics (PK/PD) properties (e.g., duration of action, maximum concentration observed (Cₘₐₓ), time-to-onset, area under the curve (AUC)), insulin can be further characterized as a rapid-acting insulin, a short-acting insulin, an intermediate-acting insulin, a long-acting insulin, and a pre-mixed insulin.

Insulin also includes monomeric and oligomeric forms, such as dimeric and hexameric forms. Insulin can exist as a monomer as it circulates in the plasma, and it also binds to its receptor while in a monomeric form. Insulin formulations (or insulin analog formulations) containing a predominance of protein molecules in the form of monomers and dimers ordinarily have a strong tendency to aggregate and form inactive fibrils. Insulin hexamers are too large to be absorbed, and so hexameric insulin formulations must disassemble into dimers or monomers before the insulin can be absorbed and function in the body. The active form of insulin in the blood stream is the monomeric form.

In some embodiments, the composition includes about 0.17 wt % to about 1.75 wt %, about 0.17 wt % to about 0.7 wt %, about 0.17 wt % to about 0.35 wt %, about 0.35 wt % to about 1.75 wt %, or about 0.35 wt % to about 0.7 wt % of the insulin or the analog thereof. In some embodiments, the composition includes about 0.17 wt %, about 0.35 wt %, about 0.7 wt %, or about 1.75 wt % of the insulin or the analog thereof. In some embodiments, the composition includes 0.17 wt % to 1.75 wt %, 0.17 wt % to 0.7 wt %, 0.17 wt % to 0.35 wt %, 0.35 wt % to 1.75 wt %, or 0.35 wt % to 0.7 wt % of the insulin or the analog thereof. In some embodiments, the composition includes 0.17 wt %, 0.35 wt %, 0.7 wt %, or 1.75 wt % of the insulin or the analog thereof.

The concentration of the insulin or the insulin analog in the composition can be measured by international units (U). In general, when applied to insulin, one international unit equals 0.0347 mg of insulin. In some embodiments, the concentration of the insulin or the insulin analog in the composition is about U50 (that is, 50 units of insulin/mL) to about 2000 U, about U50 to about U1000, about U50 to about U500, about U50 to about U200, about U50 to about U100, about U100 to about U500, about U400 to about U500, or about U100 to about U200. In some embodiments, the concentration of the insulin or the insulin analog in the composition is about U50, about U100, about U200, about U400, or about U500. In some embodiments, the concentration of the insulin or the insulin analog in the composition is about U400 to about U2000, about U500 to about U2000, about U600 to about U2000, about U700 to about U2000, about U800 to about U2000, about U900 to about U2000, or about U1000 to about U2000. In some embodiments, the concentration of the insulin or the insulin analog in the composition is about U400 to about U1500, about U500 to about U1500, about U600 to about U1500, about U700 to about U1500, about U800 to about U1500, about U900 to about U1500, or about U1000 to about U1500. In some embodiments, the concentration of the insulin or the insulin analog in the composition is about U400 to about U1000, about U500 to about U1000, about U600 to about U1000, about U700 to about U1000, about U800 to about U1000, or about U900 to about U1000. In some embodiments, the concentration of the insulin or the insulin analog in the composition is about U400 to about U2000, about U400 to about U1000, about U400 to about U900, about U400 to about U800, about U400 to about U700, about U400 to about U600, or about U400 to about U500.

In some embodiments, the composition includes about 1.7 mg/mL to about 17.5 mg/mL, about 1.7 mg/mL to about 7 mg/mL, about 1.7 mg/mL to about 3.5 mg/mL, about 3.5 wt % to about 17.5 mg/mL, or about 3.5 mg/mL to about 7 mg/mL of the insulin or the analog thereof. In some embodiments, the composition includes about 1.7 mg/mL, about 3.5 mg/mL, about 7 mg/mL, or about 17.5 mg/mL of the insulin or the analog thereof. In some embodiments, the concentration of the insulin or the insulin analog in the composition is U50 (that is, 50 U/mL) to U500, U50 to U200, U50 to U100, U100 to U500, or U100 to U200. In some embodiments, the concentration of the insulin or the insulin analog in the composition is U50, U100, U200, or U500. In some embodiments, the composition includes 1.7 mg/mL to 17.5 mg/mL, 1.7 mg/mL to 7 mg/mL, 1.7 mg/mL to 3.5 mg/mL, 3.5 wt % to 17.5 mg/mL, or 3.5 mg/mL to 7 mg/mL of the insulin or the analog thereof. In some embodiments, the composition includes 1.7 mg/mL, 3.5 mg/mL, 7 mg/mL, or 17.5 mg/mL of the insulin or the analog thereof.

In some embodiments, the concentration of the insulin or the insulin analog in the composition, is about U10 (0.35 mg/mL) to about U1000 (35 mg/mL). In some embodiments, the insulin concentration is about U50 to about U500. In some embodiments, the insulin concentration is about U100. In some embodiments, the insulin concentration is about U200. In some embodiments, the insulin concentration is about U300. In some embodiments, the insulin concentration is about U400. In some embodiments, the insulin concentration is about U500 to about U1000. In some embodiments, the insulin concentration is about U500. In some embodiments, the insulin concentration is about U600. In some embodiments, the insulin concentration is about U700. In some embodiments, the insulin concentration is about U800. In some embodiments, the insulin concentration is about U900. In some embodiments, the insulin concentration is about U1000. In some embodiments, the insulin concentration is about U1000 to about U2000. In some embodiments, the insulin concentration is about U1000 to about U1500. In some embodiments, the insulin concentration is about U1500 to about U2000.

In some embodiments, the insulin or the analog thereof is selected from insulin lispro, HUMALOG® (fast-acting insulin lispro), insulin glargine, LANTUS® (insulin glargine), insulin detemir, LEVEMIR® (insulin detemir), ACTRAPID® (fast-acting human insulin), modern insulin, NOVORAPID® (insulin aspart), VELOSULIN® (human insulin), HUMULIN® M3 (a mixture of soluble insulin and isophane insulin called biphasic isophane insulin), HYPURIN® (neutral bovine insulin), INSUMAN® (recombinant human insulin), INSULATARD® (long-acting isophane human insulin), MIXTARD® 30 (a mixture of 30% soluble insulin and 70% isophane insulin), MIXTARD® 40 (a mixture of 40% soluble insulin and 60% isophane insulin), MIXTARD® 50 (a mixture of 50% soluble insulin and 50% isophane insulin), insulin aspart, insulin glulisine, insulin isophane, insulin degludec, insulin icodec, insulin zinc extended, NOVOLIN® R (human insulin), HUMULIN® R (human insulin), HUMULIN® R regular U-500 (concentrated regular insulin), NOVOLIN® N (intermediate-acting human insulin), HUMULIN® N (intermediate-acting human insulin), RELION® (over-the-counter brand of NOVOLIN® R, NOVOLIN® N, and NOVOLIN® 70/30), AFREZZA® (rapid-acting inhaled insulin), HUMULIN® 70/30 (a mixture of 70% human insulin isophane suspension and 30% human insulin injection), NOVOLIN® 70/30 (a mixture of 70% NPH, human insulin isophane suspension and 30% regular, human insulin injection), NOVOLOG® 70/30 (a mixture of 70% insulin aspart protamine suspension and 30% insulin aspart injection), HUMULIN®50/50 (a mixture of 50% human insulin isophane suspension and 50% human insulin injection), HUMALOG® Mix 75/25 (a mixture of 75% insulin lispro protamine suspension and 25% insulin lispro injection), insulin aspart protamine-insulin aspart, insulin lispro protamine-insulin lispro, human insulin NPH-human insulin regular, insulin degludec-insulin aspart, and combinations thereof. In some embodiments, the insulin or the analog thereof is a human insulin or a recombinant human insulin. In some embodiments, the insulin or the analog thereof is a non-human (e.g., primate, pig, cow, cat, dog, or rodent) insulin or a recombinant non-human insulin. In some embodiments, the insulin or the analog thereof is a purified or synthetic insulin. In some embodiments, the insulin or the analog thereof is selected from a rapid-acting insulin, a short-acting insulin, an intermediate-acting insulin, a long-acting insulin, and a pre-mixed insulin. In some embodiments, the insulin or the analog thereof is insulin lispro. In some embodiments, the insulin or the analog thereof is insulin aspart. In some embodiments, the insulin, or an analog thereof, is recombinant human insulin.

In some embodiments, no more than 50 wt %, for example, no more than 40 wt %, no more than 30 wt %, no more than 25 wt %, no more than 20 wt %, no more than 15 wt %, no more than 10 wt %, no more than 1 wt % or substantially none of the insulin or the analog thereof is present in the composition in a hexameric or higher-order association state. In some embodiments, the substantially all of the insulin or the analog thereof in the composition is present in a lower order association state, i.e., monomer, dimer, trimer or tetramer association state.

In some embodiments, at least 50 wt %, for example, at least 60 wt %, at least 70 wt %, at least 80 wt %, at least 90 wt %, at least 95 wt %, or substantially all of the insulin or the analog thereof is present in the composition in a monomeric, dimeric, or tetrameric association state.

In some embodiments, at least 25 wt %, for example, at least 30 wt %, at least 40 wt %, or at least 50 wt % of the insulin or the analog thereof is present in the composition in a monomeric association state.

### 1.1.4. Pharmaceutical Compositions

In some embodiments, the insulin composition including the acrylamide-based copolymer and preservative (e.g., as described above) is a pharmaceutical composition, and further includes a pharmaceutically acceptable excipient. The term “pharmaceutical composition” refers to a preparation which is in such form as to permit the biological activity of an active ingredient contained therein to be administered to, and effective in treating, a subject, and which contains no additional components which are unacceptably toxic to the subject.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids; antimicrobials; antioxidants; buffers; chelating agents; complexing agents; monosaccharides; disaccharides and other carbohydrates; emulsifying agents; salt-forming counterions; solvents; sugar alcohols; suspending agents; surfactants or wetting agents; stability enhancing agents; tonicity enhancing agents; delivery vehicles; diluents; other excipients and/or pharmaceutical adjuvants. Neutral buffered saline or saline mixed with conspecific serum albumin are examples of appropriate diluents. The composition may be formulated as a lyophilizate using appropriate excipient solutions as diluents. Suitable components are nontoxic to recipients at the dosages and concentrations employed. Further examples of components that may be employed in pharmaceutical formulations are presented in Remington's Pharmaceutical Sciences, 16th Ed. (1980) and 20th Ed. (2000), Mack Publishing Company, Easton, PA.

In some embodiments, the compositions described herein further include a buffer. In some embodiments, the buffer includes phosphate, citrate, acetate, TRIS, succinate, other organic acids, or histidine. In some embodiments, the buffer includes one or more phosphate salts. In some embodiments, the buffer includes sodium phosphate.

In some embodiments, the composition is an aqueous formulation. In certain embodiments, such aqueous formulations have a pH of 3 to 7.5, such as a pH of 4 to 6.5.

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the polypeptide. For example, suitable compositions may be water for injection, physiological saline solution for parenteral administration.

In some embodiments, the pharmaceutical composition is formulated for intravenous, intramuscular, or subcutaneous administration. For intravenous, cutaneous or subcutaneous injection, or injection at the site of affliction, the active ingredient will be in the form of a parenterally acceptable aqueous formulation, such as a solution, which is pyrogen-free and has suitable pH, isotonicity, and stability. Those of relevant skill in the art are well able to prepare suitable aqueous formulations using, for example, isotonic vehicles such as Sodium Chloride Injection, Ringer's Injection, Lactated Ringer's Injection. Stabilizers, buffers, antioxidants and/or other additives can be included, as required.

### 1.2. Methods of Use

Aspects of this disclosure include methods of using the compositions that include administering a composition of the present disclosure including insulin or an analog thereof, a polyacrylamide-based copolymer and a preservative (e.g., as described herein) to a subject in need thereof. In some embodiments, administration is via subcutaneous injection. In some embodiments, administration is via intravenous injection. In some embodiments, the composition is administered via a pump. In some embodiments, the pump is an infusion pump. In some embodiments, the composition is administered via an artificial pancreas closed-loop system. In some embodiments, the composition is administered via an automated insulin delivery system. In some embodiments, the elevated glucose level is associated with insulin deficiency in the subject. In some embodiments, the subject has been diagnosed with Type 1 diabetes. In some embodiments, the subject has been diagnosed with Type 2 diabetes. In some embodiments, the subject is non-diabetic. In some embodiments, the subject has experienced trauma, surgery, or both. In some embodiments, the composition administered includes a therapeutically effective amount of insulin on an analog thereof.

As used herein, the term “subject” means a mammalian subject. Exemplary subjects include humans, monkeys, apes, dogs, cats, mice, rats, cows, horses, camels, goats, rabbits, and sheep. In certain embodiments, the subject is a human.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment can include one or more of reducing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “therapeutically effective amount” or “effective amount” refers to an amount of insulin or pharmaceutical composition that, when administered to a subject, is effective to treat a disease or disorder. The exact dose or amount will depend on the purpose of the treatment and will typically be ascertainable by one skilled in the art using known techniques (see, e.g., Lloyd (1999) The Art, Science and Technology of Pharmaceutical Compounding).

Provided in the present disclosure are methods of administering an insulin composition (e.g., as described herein) for managing the blood glucose level in a subject in need thereof. In some embodiments, provided is a method of treating elevated blood glucose levels in a subject in need thereof. In some embodiments, the elevated blood glucose level is associated with insulin deficiency. In some embodiments, the elevated blood glucose level is associated with intake of food (e.g., during a mealtime). For example, provided is a method to offset the rise in glucose levels that can accompany ingesting macronutrients that raise glucose, such as carbohydrates (i.e., mealtime insulin). In some embodiments, managing the blood glucose level includes reducing the glucose level in a subject in need thereof. In some embodiments, managing the blood glucose level includes regulating the blood glucose level in a subject diagnosed with Type 1 or Type 2 diabetes. In some embodiments, managing the blood glucose level includes reducing glucose levels during mealtime. The term “insulin deficiency,” as used herein, refers to reduced insulin levels and/or reduced insulin sensitivity relative to metabolic demand. A subject with insulin deficiency includes, but is not limited to, a subject with diabetes, including, but not limited to, Type 1 diabetes, Type 1.5 diabetes, Type 2 diabetes, gestational diabetes mellitus, and diabetes post-pancreatectomy, a subject with hyperglycemia, and a subject with transient hyperglycemia, such as transient hyperglycemia from stress in an otherwise non-diabetic subject, for example, during hospitalization. In humans, blood sugar levels less than 100 mg/dL after fasting for at least eight hours and less than 140 mg/dL two hours after eating are deemed normal. A human subject with the normal blood sugar levels described above are considered to be non-diabetic.

### 1.3. Definitions

In this disclosure, the terms “a,” “an,” and “the” are used to include one or more than one unless the context clearly dictates otherwise. The term “or” is used to refer to a nonexclusive “of” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used in the present disclosure can allow for a degree of variability in a value or range that is within 5% of a stated value or of a stated limit of a range.

In the methods described in the present disclosure, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The term “polymer” refers to a substance or material consisting of repeating monomer subunits. The term “co-polymer” refers to a polymer composed of two or more distinct repeating monomer subunits.

The term “water-soluble carrier monomer” refers to an acrylamide monomer species that is the water-soluble species within the polyacrylamide-based copolymer. In some embodiments, the water-soluble carrier monomer is the predominant species within the polyacrylamide-based copolymer. In some embodiments, the water-soluble carrier monomer imparts aqueous solubility to the copolymer. In some embodiments, the water-soluble carrier monomer within the polyacrylamide-based copolymer provides an inert barrier at the interface of an aqueous formulation to prevent protein-protein interactions. In some embodiments, the interface is an air-water interface. In some embodiments, the interface is an enclosure-water interface, including, but not limited to, a glass-water interface, a rubber-water interface, a plastic-water interface, or a metal-water interface. In some embodiments, the interface is an oil-water interface. In some embodiments, the interface is an interface between a liquid and tubing. In some embodiments, the interface is an interface between a liquid and a catheter. In some embodiments, the enclosure-water interface is in a pump system. In some embodiments, the enclosure-water interface is in a closed-loop system. In some embodiments, the water-soluble carrier monomer is nonionic. Examples of water-soluble carrier monomers include, but are not limited to, acrylamide (AM), N-(3-methoxypropoyl)acrylamide (MPAM), 4-acryloylmorpholine (MORPH), N,N-dimethylacrylamide (DMA), and N-hydroxyethyl acrylamide (HEAM).

The term “functional dopant monomer,” as used herein, refers to an acrylamide monomer species that has physicochemical properties (e.g., hydrophobicity, charge) different from those of the water-soluble carrier monomer. In some embodiments, the functional dopant monomer within the polyacrylamide-based copolymer promotes association of the polymers to an interface; such interfaces can include, but are not limited to, polymer-air-water interface interactions, polymer-protein interactions, polymer-peptide interactions, polymer-micelle interactions, polymer-liposome interactions, and polymer-lipid nanoparticle interactions. The functional dopant monomer can act as a stabilizing moiety to facilitate interactions with biomolecules, for example, proteins, peptides, antibodies, antibody-drug conjugates, nucleic acids, lipid particles, and combinations thereof (e.g., to prevent aggregation of the biomolecules). The functional dopant monomers can be further classified into hydrogen-bonding, ionic, hydrophobic, and aromatic monomers based on their chemical composition. Typically, the functional dopant monomers are copolymerized at a lower weight percentage as compared to the water-soluble carrier monomers.

The term “polymerization” refers to the process in which monomer molecules undergo a chemical reaction to form polymeric chains or three-dimensional networks. Different types of polymerization reactions are known in the art, for example, addition (chain-reaction) polymerization, condensation polymerization, ring-opening polymerization, free radical polymerization, controlled radical polymerization, atom transfer radical polymerization (ATRP), single-electron transfer living radical polymerization (SET-LRP), reversible addition-fragmentation chain transfer (RAFT) polymerization, nitroxide-mediated polymerization (NMP), and emulsion polymerization. In some embodiments, the copolymers of the present disclosure are prepared using RAFT polymerization.

The term “degree of polymerization” (DP) refers to the number of monomer units in a polymer. It is calculated by dividing the average molecular weight of a polymer sample by the molecular weight of the monomers. As defined herein, the average molecular weight of a polymer can be represented by the number-averaged molecular weight (Mn), the weight-average molecular weight (Mw), the Z-average molecular weight (Mz) or the molecular weight at the peak maxima of the molecular weight distribution curve (Mp). The average molecular weight of a polymer can be determined by a variety of analytical characterization techniques known to those skilled in the art, for example, gel permeation chromatography (GPC), static light scattering (SLS) analysis, multi-angle laser light scattering (MALLS) analysis, nuclear magnetic resonance spectroscopy (NMR), intrinsic viscometry (IV), melt flow index (MFI), and matrix-assisted laser desorption/ionization mass spectrometry (MALDI-MS), and combinations thereof. Degree of polymerization can also be determined experimentally using suitable analytical methods known in the art, such as nuclear magnetic spectroscopy (NMR), Fourier Transform infrared spectroscopy (FT-IR) and Raman spectroscopy.

Used in reference to insulin or insulin-analog-containing formulations, the term “duration of action” refers to “time to 50% of peak down,” that is, the time after administration of the formulation at which serum concentration of the insulin or insulin analog has decreased to 50% of the peak serum concentration. See, e.g., Maikawa et al., Adv. Sci. 8(21):2101575 (2021).

As used herein, the term “substantially free from” a particular substance, in the context of a composition, refers to the absence of the substance in the composition or the presence of the substance in the composition only in amounts that do not measurably impact the ability of the composition to perform its intended function, such as, for certain of the compositions disclosed herein, a method of treating an elevated glucose level in a subject in need thereof or a method of managing a blood glucose level in a subject in need thereof.

### Additional Embodiments

The disclosure is further described by the following non-limiting clauses:

Clause 1. A composition comprising: a polyacrylamide-based copolymer comprising: a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof, and a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), N-phenylacrylamide (PHE), and combinations thereof; a preservative; and insulin, or an analog thereof.

Clause 2. The composition of clause 1, wherein the preservative is selected from meta-cresol, phenoxyethanol, methylparaben, propylparaben, and combinations thereof.

Clause 3. The composition of clause 1 or 2, wherein the water-soluble carrier monomer is selected from MORPH, MPAM, and combinations thereof.

Clause 4. The composition of clause 3, wherein the water-soluble carrier monomer comprises MORPH.

Clause 5. The composition of clause 3, wherein the water-soluble carrier monomer comprises MPAM.

Clause 6. The composition of any one of clauses 1-5, wherein the functional dopant monomer is selected from AMP, TMA, TBA, PHE, and combinations thereof.

Clause 7. The composition of clause 6, wherein the functional dopant monomer comprises AMP.

Clause 8. The composition of clause 6, wherein the functional dopant monomer comprises TMA.

Clause 9. The composition of clause 6, wherein the functional dopant monomer comprises TBA.

Clause 10. The composition of clause 6, wherein the functional dopant monomer comprises PHE.

Clause 11. The composition of any one of clauses 1-5, wherein the functional dopant monomer is selected from DEA, TRI, PHE, NIP, and combinations thereof.

Clause 12. The composition of clause 11, wherein the functional dopant monomer comprises DEA.

Clause 13. The composition of clause 11, wherein the functional dopant monomer comprises TRI.

Clause 14. The composition of clause 11, wherein the functional dopant monomer comprises PHE.

Clause 15. The composition of clause 11, wherein the functional dopant monomer comprises NIP.

Clause 16. The composition of clause 1, wherein: the water-soluble carrier monomer is selected from MPAM, MORPH, and combinations thereof, and the functional dopant monomer is selected from NIP, PHE, and combinations thereof.

Clause 17. The composition of clause 16, wherein: the water-soluble carrier monomer comprises MPAM; and the functional dopant monomer comprises NIP.

Clause 18. The copolymer of clause 16, wherein: the water-soluble carrier monomer comprises MPAM; and the functional dopant monomer comprises PHE.

Clause 19. The composition of clause 16, wherein: the water-soluble carrier monomer comprises MORPH; and the functional dopant monomer comprises NIP.

Clause 20. The composition of clause 16, wherein: the water-soluble carrier monomer comprises MORPH; and the functional dopant monomer comprises PHE.

Clause 21. The composition of any one of clauses 1-20, wherein the copolymer comprises 70 wt % to 98 wt % of the water-soluble carrier monomer.

Clause 22. The composition of any one of clauses 1-21, wherein the copolymer comprises 2 wt % to 30 wt % of the functional dopant monomer.

Clause 23. The composition of clause 22, wherein the copolymer comprises: 70 wt % to 85 wt % of MORPH; and 15 wt % to 30 wt % of NIP.

Clause 24. The composition of clause 23, wherein the copolymer comprises: 74 wt % to 80 wt % of MORPH; and 20 wt % to 26 wt % of NIP.

Clause 25. The composition of clause 23, wherein the copolymer comprises about 77 wt % of MORPH.

Clause 26. The composition of any one of clauses 1-25, wherein a degree of polymerization of the copolymer is 10 to 500.

Clause 27. The composition of clause 26, wherein a degree of polymerization of the copolymer is 20 to 200.

Clause 28. The composition of any one of clauses 1-27, wherein a number-average molecular weight of the copolymer is 2,000 g/mol to 10,000 g/mol.

Clause 29. The composition of clause 28, wherein a number-average molecular weight of the copolymer is 2,000 g/mol to 6,000 g/mol.

Clause 30. The composition of clause 29, wherein a number-average molecular weight of the copolymer is 3,000 g/mol to 5,000 g/mol.

Clause 31. The composition of any one of clauses 1-30, wherein the composition comprises 0.001 wt % to 1 wt % of the copolymer.

Clause 32. The composition of clause 31, wherein the composition comprises 0.005 wt % to 0.5 wt % of the copolymer.

Clause 33. The composition of clause 32, wherein the composition comprises 0.01 wt % of the copolymer.

Clause 34. The composition of any one of clauses 1-33, wherein the composition comprises 0.01 wt % to 2 wt % of the preservative.

Clause 35. The composition of clause 34, wherein the composition comprises 0.05 wt % to 1.5 wt % of the preservative.

Clause 36. The composition of any one of clauses 1-35, wherein the preservative comprises phenoxyethanol.

Clause 37. The composition of any one of clauses 1-36, wherein the preservative comprises phenoxyethanol and meta-cresol.

Clause 38. The composition of any one of clauses 1-35, wherein the preservative comprises methylparaben and propylparaben.

Clause 39. The composition of clause 36, wherein the composition comprises 0.2 wt % to 1.5 wt % of phenoxyethanol.

Clause 40. The composition of clause 39, wherein the composition comprises 0.5 wt % to 1.1 wt % of phenoxyethanol.

Clause 41. The composition of clause 40, wherein the composition comprises 0.85 wt % of phenoxyethanol.

Clause 42. The composition of any one of clauses 1-37, wherein the composition comprises: 0.1 wt % or less of meta-cresol; and 0.3 wt % to 1 wt % of phenoxyethanol.

Clause 43. The composition of any one of clauses 1-35 and 38, wherein the composition comprises: 0.05 wt % to 0.5 wt % of methylparaben; and 0.001 wt % to 0.1 wt % of propylparaben.

Clause 44. The composition of clause 43, wherein the composition comprises: 0.09 wt % to 0.2 wt % of methylparaben; and 0.005 wt % to 0.05 wt % of propylparaben.

Clause 45. The composition of clause 43 or 44, wherein the ratio of propylparaben to methylparaben is from 1:8 to 1:10.

Clause 46. The composition of clause 45, wherein the ratio of propylparaben to methylparaben is 1:9.

Clause 47. The composition of any of clauses 1-46, wherein the composition further comprises a tonicity modifier.

Clause 48. The composition of clause 47, wherein the composition comprises 0.5 wt % to 5 wt % of the tonicity modifier.

Clause 49. The composition of clause 48, wherein the composition comprises 1.5 wt % to 3 wt % of the tonicity modifier.

Clause 50. The composition of any one of clauses 47-49, wherein the tonicity modifier is glycerol, sodium chloride, or a combination thereof.

Clause 51. The composition of clause 50, wherein the composition comprises 1.5 wt % to 3 wt % of glycerol.

Clause 52. The composition of clause 51, wherein the composition comprises 2.6 wt % of glycerol.

Clause 53. The composition of any one of clauses 1-52, wherein the composition is substantially free from meta-cresol, preferably including less than 0.1 wt %, e.g., less than 0.05 wt %, less than 0.01 wt %, or less than 0.001 wt % of meta-cresol in the composition.

Clause 53. The composition of any one of clauses 1-49, wherein the composition is substantially free from zinc, preferably including less than 0.1 wt % of zinc, less than 0.05 wt %, less than 0.01 wt %, or less than 0.001 wt % of zinc in the composition.

Clause 54. The composition of any one of clauses 1-53, wherein the composition comprises 0.17 wt % to 1.75 wt % of the insulin or the analog thereof.

Clause 55. The composition of clause 54, wherein the composition comprises 0.35 wt % of the insulin or the analog thereof.

Clause 56. The composition of any one of clauses 1-55, wherein the insulin or the analog thereof is selected from porcine insulin, bovine insulin, feline insulin, human insulin, recombinant insulin, insulin lispro, HUMALOG®, insulin glargine, LANTUS®, insulin detemir, LEVEMIR®, ACTRAPID®, modern insulin, NOVORAPID®, VELOSULIN®, HUMULIN® M3, HYPURIN®, INSUMAN®, INSULATARD®, MIXTARD® 30, MIXTARD® 40, MIXTARD® 50, insulin aspart, insulin glulisine, insulin isophane, insulin degludec, insulin icodec, insulin zinc extended, NOVOLIN® R, HUMULIN® R, HUMULIN® R regular U-500, NOVOLIN® N, HUMULIN® N, RELION®, AFREZZA®, HUMULIN® 70/30, NOVOLIN® 70/30, NOVOLOG® 70/30, HUMULIN® 50/50, HUMALOG® mix 75/25, insulin aspart protamine-insulin aspart, insulin lispro protamine-insulin lispro, human insulin NPH-human insulin regular, and insulin degludec-insulin aspart.

Clause 57. The composition of any one of clauses 1-55, wherein the insulin or the analog thereof is insulin aspart.

Clause 58. The composition of any one of clauses 1-55, wherein the insulin or the analog thereof is human insulin.

Clause 59. The composition of any one of clauses 1-55, wherein the insulin or the analog thereof is insulin lispro.

Clause 60. The composition of clause 59, wherein the composition comprises 0.17 wt % to 1.75 wt % of insulin lispro.

Clause 61. The composition of clause 60, wherein the composition comprises 0.35 wt % of insulin lispro.

Clause 62. The composition of any one of clauses 1-61, wherein the pH of the composition is 4 to 8.

Clause 63. The composition of any one of clauses 1-62, wherein the composition is aqueous.

Clause 64. The composition of any one of clauses 1-63, wherein the composition further comprises a buffer.

Clause 65. The composition of clause 64, wherein the buffer comprises phosphate, citrate, acetate, TRIS, histidine, or any combination thereof.

Clause 66. The composition of clause 65, wherein the buffer comprises a phosphate.

Clause 67. The composition of clause 1, comprising: 0.001 wt % to 1 wt % of a polyacrylamide-based copolymer comprising: 70 wt % to 98 wt % of a water-soluble carrier monomer selected from MPAM, MORPH, DMA, HEAM, and AM; and 2 wt % to 30 wt % of a functional dopant monomer selected from TRI, AMP, TMA, NIP, DEA, TBA, and PHE; 0.01 wt % to 2 wt % of a preservative selected from meta-cresol, phenoxyethanol, methylparaben, and propylparaben; and 0.17 wt % to 0.7 wt % of insulin, or an analog thereof.

Clause 68. The composition of clause 67, wherein: the water-soluble carrier monomer is selected from MPAM, and MORPH; and the functional dopant monomer is selected from NIP, and PHE.

Clause 69. The composition of clause 68, wherein the polyacrylamide-based copolymer comprises: 70 wt % to 85 wt % of MORPH; and 15 wt % to 30 wt % of NIP.

Clause 70. The composition of any one of clauses 67-69, further comprising 0.5 wt % to 5 wt % of a tonicity modifier.

Clause 71. The composition of clause 70, wherein the tonicity modifier is glycerol.

Clause 72. The composition of any one of clauses 67-71, wherein the insulin or the analog thereof is selected from human insulin, insulin aspart, and insulin lispro.

Clause 73. The composition of clause 69, comprising: 0.01 wt % of a polyacrylamide-based copolymer comprising: about 77 wt % of MORPH; and about 23 wt % of NIP.

Clause 74. The composition of clause 73, comprising: 0.85 wt % of phenoxyethanol; 2.6 wt % of glycerol; and 0.35 wt % of insulin lispro.

Clause 75. The composition of clause 73, comprising: 0.64 wt % of phenoxyethanol; 0.064 wt % of meta-cresol; 2.6 wt % of glycerol; and 0.35 wt % of insulin lispro.

Clause 76. The composition of clause 1, comprising: 0.15 wt % in total of methylparaben and propylparaben; 2.6 wt % of glycerol; and 0.35 wt % of insulin lispro.

Clause 77. The composition of clause 76, comprising: 0.135 wt % methylparaben; and 0.015 wt % of propylparaben.

Clause 78. The composition of any one of clauses 67-77, wherein the composition is substantially free from zinc, preferably including less than 0.1 wt % of zinc, less than 0.05 wt %, less than 0.01 wt %, or less than 0.001 wt % of zinc in the composition.

Clause 79. The composition of any one of clauses 1-78, wherein no more than 50 wt % of the insulin or the analog thereof is present in the composition in a hexameric or higher-order association state.

Clause 80. The composition of any one of clauses 1-79, wherein no more than 25 wt % of the insulin or the analog thereof is present in the composition in a hexameric or higher-order association state.

Clause 81. The composition of any one of clauses 1-80, wherein substantially none of the insulin or the analog thereof is present in the composition in a hexameric or higher-order association state.

Clause 82. The composition of any one of clauses 1-78, wherein at least 50 wt % of the insulin or the analog thereof is present in the composition in a monomeric, dimeric, trimeric, or tetrameric association state.

Clause 83. The composition of any one of clauses 1-78, wherein at least 90 wt % of the insulin or the analog thereof is present in the composition in a monomeric, dimeric, trimeric, or tetrameric association state.

Clause 84. The composition of any one of clauses 1-78, wherein at least 25 wt % of the insulin or the analog thereof is present in the composition in a monomeric association state.

Clause 85. The composition of any one of clauses 1-78, wherein at least 50 wt % of the insulin or the analog thereof is present in the composition in a monomeric association state.

Clause 86. The composition of any one of clauses 1-85, having a peak action of 30 minutes or less.

Clause 87. The composition of clause 86, having a peak action of 20 minutes or less.

Clause 88. The composition of any one of clauses 1-87, having a duration of action of 150 minutes or less.

Clause 89. A method of treating an elevated glucose level in a subject in need thereof, comprising administering to the subject a therapeutically effective amount of a composition of any one of clauses 1-88, wherein the elevated glucose level is associated with insulin deficiency in the subject.

Clause 90. A method of managing a blood glucose level in a subject in need thereof, comprising administering to the subject a therapeutically effective amount of a composition of any of clauses 1-88.

Clause 91. The method of clause 89 or 90, wherein the composition is administered via an infusion pump or an artificial pancreas closed-loop system.

Clause 92. Use of a composition for the manufacture of a medicament for treating an elevated glucose level in a subject in need thereof, wherein the composition is of any one of clauses 1-88, and wherein the elevated glucose level is associated with insulin deficiency in the subject.

Clause 93. Use of a composition for the treatment of an elevated glucose level in a subject in need thereof, wherein the composition is of any one of clauses 1-88, and wherein the elevated glucose level is associated with insulin deficiency in the subject.

Clause 94. Use of a composition for the manufacture of a medicament for managing a blood glucose level in a subject in need thereof, wherein the composition is of any one of clauses 1-88.

Clause 95. Use of a composition for the management of a blood glucose level in a subject in need thereof, wherein the composition is of any one of clauses 1-88.

### EXAMPLES

The disclosure is further described in the following examples, which are not intended to limit the scope of the invention as set forth in the claims.

### Example 1—Preparation of Compositions

### Materials

The amphiphilic acrylamide copolymer excipient acryloylmorpholine_{77%}-N-isopropylacrylamide_{23%}(MoNi_{23%}) was prepared according to protocols described by Mann et al., Sci. Transl. Med 12, eaba6676 (2020) and manuscript supplementary materials. Characterization of MoNi_{23%} molecular weight can be found in Table 1. The target wt % of the monomers in the MoNi_{23%} co-polymer is based upon the expected wt % in the copolymer product derived from the amounts of precursor monomers.

TABLE 1
MoNi copolymer excipient characterization. Mn (number average molecular
weight), Mw (weight average molecular weight), and Ð (dispersity)
determined via DMF size exclusion chromatography calibrated using
polyethylene glycol standards for HEAM, MPAM, MORPH, and DMA.

| | | wt. % by | | | wt. % by | | | |
|---|---|---|---|---|---|---|---|---|
| Carrier | wt. % | NMR | Dopant | wt. % | NMR | Mₙ^{a} | M_{w}^{a} | |
| Monomer | (Target) | (Exp) | Monomer | (Target) | (Exp) | (Da) | (Da) | Ð^{a} |
| MORPH | 77 | 74.5 ^{b} | NIP | 23 | 25.5 ^{b} | 3200 | 3800 | 1.19 |

^{a}Determined using Size Exclusion Chromatography calibrated using polyethylene glycol samples.
^{b} Weight percentages difficult to determine due to overlapping spectra. Weight percentages estimated from post-precipitated NMR spectra by measuring the more resolved left half of the peak of N-isopropylacrylamide (δ = 4.0, 0.5 H), doubling it, and subtracting it from the unresolved peaks of Mo and Ni (δ = 3.2-4.2, 7H (Mo) 1H (Ni)).

Humalog (Eli Lilly) and Humulin R (Eli Lilly) were purchased and used as received. For zinc-free lispro and zinc-free regular human insulin, Zinc(II) was removed from the commercial insulin formulations through competitive binding by addition of ethylenediaminetetraacetic acid (EDTA), which exhibits a dissociation binding constant approaching attomolar concentrations (K_{D}˜10⁻¹⁸ M). EDTA was added to formulations (4 molar eq with respect to zinc) to sequester zinc from the formulation and then lispro was isolated using PD MidiTrap G-10 gravity columns (GE Healthcare) to buffer exchange into water. The solution was then concentrated using Amicon Ultra 3K centrifugal units (Millipore) and reformulated with glycerol, phenoxyethanol, meta-cresol, methylparaben, and/or propylparaben in 10 mM phosphate buffer (pH=7.4) at an insulin concentration of 3.45 mg/mL (100 U/mL) (excipient concentrations specified in Tables 2-3). All other reagents were purchased from Sigma-Aldrich unless otherwise specified.

TABLE 2
Excipient Concentrations for exemplary insulin lispro formulations.

| Formulation | Humalog | | lispro-1 | | lispro-2 | | lispro-3 | | lispro-4 | |
|---|---|---|---|---|---|---|---|---|---|---|
| zinc | 0.0019 | wt. % | — | | — | | — | | — | |
| glycerol | 1.6 | wt. % | 2.6 | wt. % | 2.6 | wt. % | 2.6 | wt. % | 2.6 | wt. % |

| | | | | | | | | |
|---|---|---|---|---|---|---|---|---|
| meta-cresol | 0.32 | wt. % | 0.32 | wt. % | 0.064 | wt. % | — | — |

| phenoxyethanol | — | — | | 0.64 | wt % | 0.85 | wt. % | — | |
|---|---|---|---|---|---|---|---|---|---|
| methylparaben | — | — | | — | | — | | 0.137 | wt. % |
| propylparaben | — | — | | — | | — | | 0.015 | wt. % |
| MoNi_{23%} | — | 0.01 | wt. % | 0.01 | wt. % | 0.01 | wt. % | 0.01 | wt. % |

TABLE 3
Excipient concentrations for exemplary regular human insulin formulations

| Formulation | Humulin | | RHI-1 | | RHI-2 | | RHI-3 | | RHI-4 | |
|---|---|---|---|---|---|---|---|---|---|---|
| zinc | 0.0019 | wt. % | — | | — | | — | | — | |
| glycerol | 1.6 | wt. % | 2.6 | wt. % | 2.6 | wt. % | 2.6 | wt. % | 2.6 | wt. % |

| | | | | | | | | |
|---|---|---|---|---|---|---|---|---|
| meta-cresol | 0.32 | wt. % | 0.32 | wt. % | 0.064 | wt. % | — | — |

| phenoxyethanol | — | — | | 0.64 | wt % | 0.85 | wt. % | — | |
|---|---|---|---|---|---|---|---|---|---|
| methylparaben | — | — | | — | | — | | 0.137 | wt. % |
| propylparaben | — | — | | — | | — | | 0.015 | wt. % |
| MoNi_{23%} | — | 0.01 | wt. % | 0.01 | wt. % | 0.01 | wt. % | 0.01 | wt. % |

### Analytical Ultracentrifugation

Insulin compositions were prepared at 3.45 mg/mL (100 U/mL) with excipients as described in Tables 2-3. Analytical ultracentrifugation was performed by HTL Biosolutions. Briefly, samples were analyzed using a ProteomeLab XL-I analytical ultracentrifuge with 2-channel charcoal-epon centerpieces with 12 mm optical pathlength and an 8-hole An-50 Ti analytical rotor (20° C., 45,000 rpm). Data were analyzed by HTL Biosolutions using SEDFIT version 16.2b and Gussi 1.3.1 software. SEDFIT (version 16.2b) developed by Peter Schuck at the N.I.H. was used for data analysis. The scans 1-200 were included for data analysis using the c(s) method. The f/fₒ values and meniscus position were fitted to find the best overall fit of the data for each sample. A maximum entropy regularization probability of 0.683 (1 σ) was used, and time-independent noise was removed. The continuous c(s) distribution model was used for data analysis of all samples as the signal from polymer is less than 3% of the total signal (the polymer concentration in each sample is 0.1 mg/mL and the insulin concentration is 3.5 mg/mL).

The following parameter was used for the AUC-SV distribution analysis:
Partial specific volume: 0.73 mL/g
Density: 1.0000 mg/mL
Viscosity: 0.01002 P

Analytical ultracentrifugation is a useful method to determine insulin association states as sedimentation coefficients can be used to identify different association states and sedimentation velocity can be used to estimate the relative amounts of each species present. Since sedimentation coefficients are dependent on the molecular shape and mass of proteins, association state can be predicted when the monomer sedimentation coefficient is known. If it is assumed that the aggregate shape is similar to that of the monomer then its stoichiometry, N, will be given by (sN/s1)^{3/2}, where sN is the sedimentation coefficient of the N-mer and s1 is the sedimentation coefficient of monomer. Table 4 describes the “rule of thumb” ranges of sedimentation coefficients provided by HTL Biosolutions (relative to the monomer sedimentation coefficient) used to assign association states.

TABLE 4
Sedimentation Coefficients Corresponding
to Insulin Association States

| Insulin Association | Ratio of oligomer sedimentation coefficient to |
|---|---|
| State | monomer sedimentation coefficient* |
| Monomer | ~1 |
| Dimer/Trimer | 1.45-1.86 |
| Tetramer | 2-2.26 |
| Hexamer | 2.67-2.97 |
| Higher Order | >3.46 |
| (Octamer+) | |

*These were provided by HTL Biosolutions as “rule of thumb” values. Assignment of association states were rounded to the nearest range of sedimentation coefficients.

Observed sedimentation coefficients are dependent on solvent/buffer density, viscosity, and temperature, as well as transient species (those with lifetimes of less than ˜1 h) can cause a shift in sedimentation coefficients. When necessary, association state was assigned by rounding to the nearest range of sedimentation coefficients (assignment can be found in Tables 5 and 6).

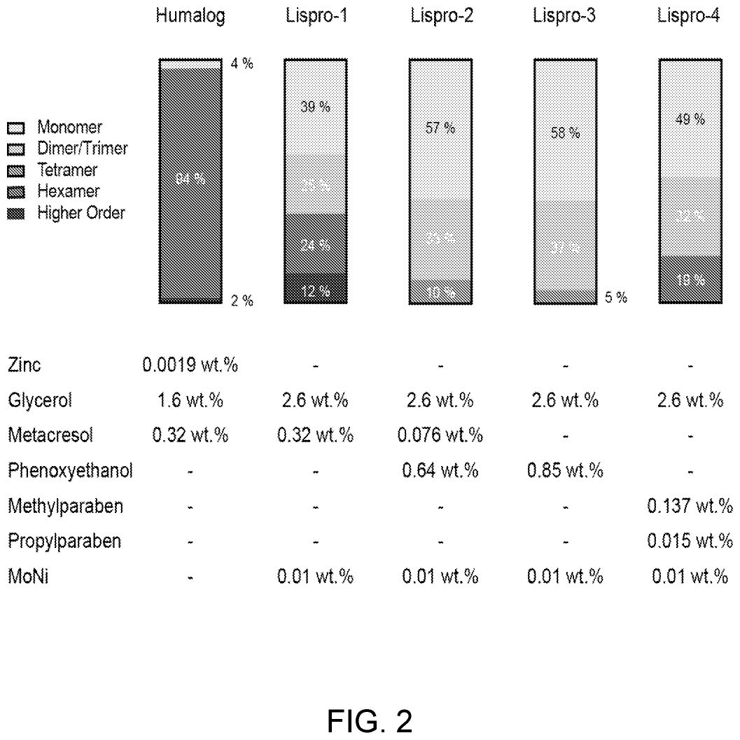

Using analytical ultracentrifugation, sedimentation coefficients were used to estimate the ratios of insulin association states when formulated with different antimicrobial preservatives in the absence of zinc. Commercial Humalog is shown as a control. In lispro 1-4 formulations, EDTA was added to lispro (4:1 EDTA:zinc) to chelate zinc and then the EDTA zinc complex was removed with a desalting column. Meta-cresol, phenoxyethanol, a mixture of meta-cresol and phenoxyethanol, and a mixture of methylparaben and propylparaben were tested as preservatives. It should be noted that higher ordered structures as reported here is thought to be reversible aggregates such as octamers, rather than high molecular weight polymer or amyloid fibril species. FIG. 2 and Table 5 illustrate the insulin lispro association states for the various lispro formulations with differing amounts of excipients.

TABLE 5
Ratio of insulin lispro association states by
insulin sedimentation coefficients. See FIG. 2.
Humalog

| | | | | | |
|---|---|---|---|---|---|
| sed c(s) | 1.14 | 2.86 | 5.83 | 8.23 | 8.94 |
| % peak | 3.6 | 94.3 | 1.1 | 0.4 | 0.6 |
| classification | monomer | hexamer | higher | higher | higher |
| | | | order | order | order |

lispro-1

| | | | | | |
|---|---|---|---|---|---|
| sed c(s) | 0.85 | 1.84 | 3.03 | 3.58 | >3.6 |
| % peak | 38.9 | 24.5 | 24.5 | 12 | 0.1 |
| classification | monomer | dimer/trimer | hexamer | higher | higher |
| | | | | order | order |

lispro-2

| | | | |
|---|---|---|---|
| sed c(s) | 0.89 | 1.62 | 2.37 |
| % peak | 57.3 | 33.1 | 9.6 |
| classification | monomer | dimer/trimer | tetramer |

lispro-3

| | | | | |
|---|---|---|---|---|
| sed c(s) | 0.81 | 1.4 | 2.3 | >2.3 |
| % peak | 57.8 | 36.8 | 4.9 | 0.5 |
| classification | monomer | dimer/trimer | tetramer | hexamer |

lispro-4

| | | | | |
|---|---|---|---|---|
| sed c(s) | 0.91 | 1.57 | 2.57 | >2.6 |
| % peak | 48.4 | 32.3 | 19.3 | 0.1 |
| classification | monomer | dimer/trimer | hexamer | higher |
| | | | | order |

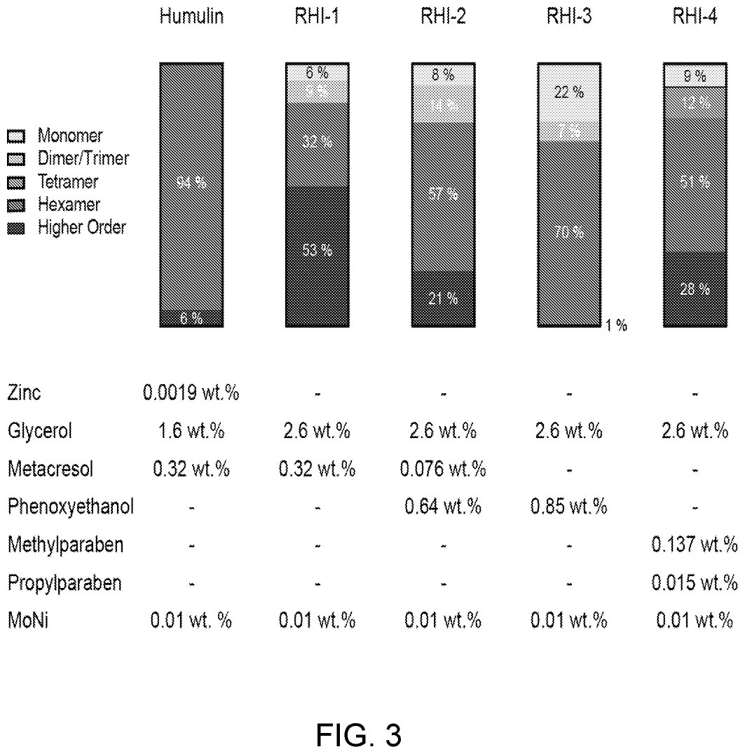

Using analytical ultracentrifugation, sedimentation coefficients were used to estimate the ratios of insulin association states when formulated with different antimicrobial preservatives in the absence of zinc. Commercial Humalog is shown as a control. In RHI-4 formulations, EDTA was added to RHI (4:1 EDTA:zinc) to chelate zinc and then the EDTA zinc complex was removed with a desalting column. Meta-cresol, phenoxyethanol, a mixture of meta-cresol and phenoxyethanol, and a mixture of methylparaben and propylparaben were tested as preservatives. It should be noted that higher ordered structures as reported here is thought to be reversible aggregates such as octamers, rather than high molecular weight polymer or amyloid fibril species. FIG. 3 and Table 6 illustrate the regular human insulin (RHI) associate states for various RHI formulations with differing compositions of excipients.

TABLE 6
Ratio of regular human insulin association states
by insulin sedimentation coefficients.
Humulin

| | | | | |
|---|---|---|---|---|
| sed c(s) | 2.8 | 5.78 | 9.14 | >9.2 |
| % peak | 93.81 | 3.29 | 0.9 | 2 |
| classification | hexamer | higher | higher | higher |
| | | order | order | order |

RHI-1

| | | | | | |
|---|---|---|---|---|---|
| sed c(s) | 0.83 | 1.85 | 3.1 | 3.61 | 4.37 |
| % peak | 6.39 | 8.44 | 31.9 | 11.36 | 41.93 |
| classification | monomer | dimer/trimer | hexamer | higher | higher |
| | | | | order | order |

RHI-2

| | | | | |
|---|---|---|---|---|
| sed c(s) | 1.02 | 1.86 | 2.83 | 3.37 |
| % peak | 8.36 | 14.09 | 56.7 | 20.86 |
| classification | monomer | dimer/trimer | hexamer | higher |
| | | | | order |

RHI-3

| | | | | |
|---|---|---|---|---|
| sed c(s) | 0.77 | 1.65 | 2.69 | >2.7 |
| % peak | 21.9 | 7.48 | 69.9 | 0.7 |
| classification | monomer | dimer/trimer | hexamer | hexamer |

RHI-4

| | | | | | | |
|---|---|---|---|---|---|---|
| sed c(s) | 0.87 | 1.96 | 2.85 | 3.06 | 3.71 | >3.8 |
| % peak | 9.17 | 11.38 | 26.89 | 24.07 | 28.36 | 0.1 |
| classification | monomer | tetramer | hexamer | hexamer | higher | higher |
| | | | | | order | order |

### In Vitro Stability

Aggregation assays used to evaluate stability were adapted from the literature. Weber et al., Proc. Natl. Acad. Sci. U.S.A. 113(5):14189-94 (2016). Briefly, formulations were aliquoted 150 μL per well (n=3/group) in a clear 96-well plate and sealed with optically clear and thermally stable seal (VWR). The plate was incubated in a microplate reader (BioTek SynergyH1 microplate reader) at 37° C. with continuous agitation (567 cpm). Absorbance readings were taken every 10 minutes at 540 nm for the duration of the experiment. The formation of insulin aggregates led to light scattering and a reduction in the transmittance of samples (time to aggregation=time to 10% change in transmittance).

### Statistics

All data is shown as mean s.e.m. unless specified otherwise. For time to aggregation data, a one-way ANOVA with a Tukey-Kramer correction for multiple comparisons was performed in GraphPad Prism 9, and each formulation was compared to all other formulations. Adjusted p values are listed in Tables 7-10 (α<0.05).

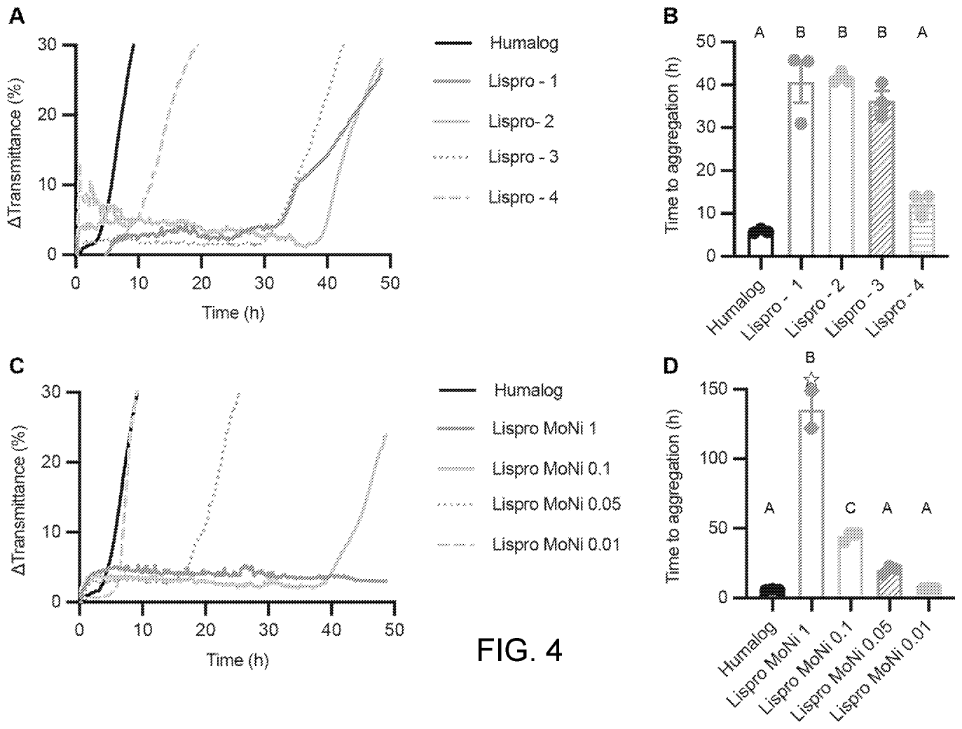

The stability of exemplary insulin lispro formulations was assessed. FIG. 4 is a set of graphs showing insulin lispro formulation stability. (Panel A) Change in transmittance traces and (Panel B) Time to aggregation of formulations with different antimicrobial preservatives: lispro—1 (meta-cresol), lispro—2 (meta-cresol+phenoxyethanol), lispro—3 (phenoxyethanol), lispro—4 (methylparaben+propylparaben). (Panel C) Change in transmittance traces and (Panel D) Time to aggregation of phenoxyethanol formulation (lispro—3) with different concentrations of MoNi_{23%} polymer excipient. Comparison of stability by aggregation times (tA), defined as the time to a change in transmittance (λ=540 nm) of 10% or greater following stressed aging (i.e., continuous agitation at 37° C.). Data shown are average transmittance traces for n=3 samples per group and error bars are standard error mean. * represents a sample that did not aggregate before the assay ended at 150 hours. A one-way ANOVA with a Tukey-Kramer correction for multiple comparisons was performed in GraphPad Prism 9, and each formulation was compared to all other formulations. Formulations connected by the same letter label are not significantly different. Adjusted p values are listed in Tables 7-8, below. α<0.05.

TABLE 7
Adjusted P Values for FIG. 4 (Panel B)

| Comparison | Mean difference | Adjusted P Value |
|---|---|---|
| lispro - 1 vs. lispro - 2 | −0.8200 | 0.9993 |
| lispro - 1 vs. lispro - 3 | 4.433 | 0.7355 |
| lispro - 1 vs. Humalog | 34.94 | <0.0001 |
| lispro - 1 vs. lispro - 4 | 28.37 | 0.0001 |
| lispro - 2 vs. lispro - 3 | 5.253 | 0.6083 |
| lispro - 2 vs. Humalog | 35.76 | <0.0001 |
| lispro - 2 vs. lispro - 4 | 29.19 | <0.0001 |
| lispro - 3 vs. Humalog | 30.50 | <0.0001 |
| lispro - 3 vs. lispro - 4 | 23.93 | 0.0004 |
| Humalog vs. lispro - 4 | −6.570 | 0.4124 |

TABLE 8
Adjusted P values for FIG. 4 (Panel D)

| | Mean | Adjusted |
|---|---|---|
| Comparison | difference | P Value |
| lispro MoNi_{23%} 1 vs. lispro MoNi_{23%} 0.1 | 91.00 | <0.0001 |
| lispro MoNi_{23%} 1 vs. lispro MoNi_{23%} 0.05 | 115.4 | <0.0001 |
| lispro MoNi_{23%} 1 vs. lispro MoNi_{23%} 0.01 | 128.4 | <0.0001 |
| lispro MoNi_{23%} 1 vs. lispro MoNi_{23%} 0 | 126.8 | <0.0001 |
| lispro MoNi_{23%} 1 vs. Humalog | 129.7 | <0.0001 |
| lispro MoNi_{23%} 0.1 vs. lispro MoNi_{23%} 0.05 | 24.43 | 0.0050 |
| lispro MoNi_{23%} 0.1 vs. lispro MoNi_{23%} 0.01 | 37.45 | 0.0001 |
| lispro MoNi_{23%} 0.1 vs. lispro MoNi_{23%} 0 | 35.78 | 0.0002 |
| lispro MoNi_{23%} 0.1 vs. Humalog | 38.67 | 0.0001 |
| lispro MoNi_{23%} 0.05 vs. lispro MoNi_{23%} 0.01 | 13.01 | 0.1792 |
| lispro MoNi_{23%} 0.05 vs. lispro MoNi_{23%} 0 | 11.35 | 0.2867 |
| lispro MoNi_{23%} 0.05 vs. Humalog | 14.24 | 0.1242 |
| lispro MoNi_{23%} 0.01 vs. lispro MoNi_{23%} 0 | −1.667 | 0.9993 |
| lispro MoNi_{23%} 0.01 vs. Humalog | 1.223 | 0.9998 |
| lispro MoNi_{23%} 0 vs. Humalog | 2.890 | 0.9908 |

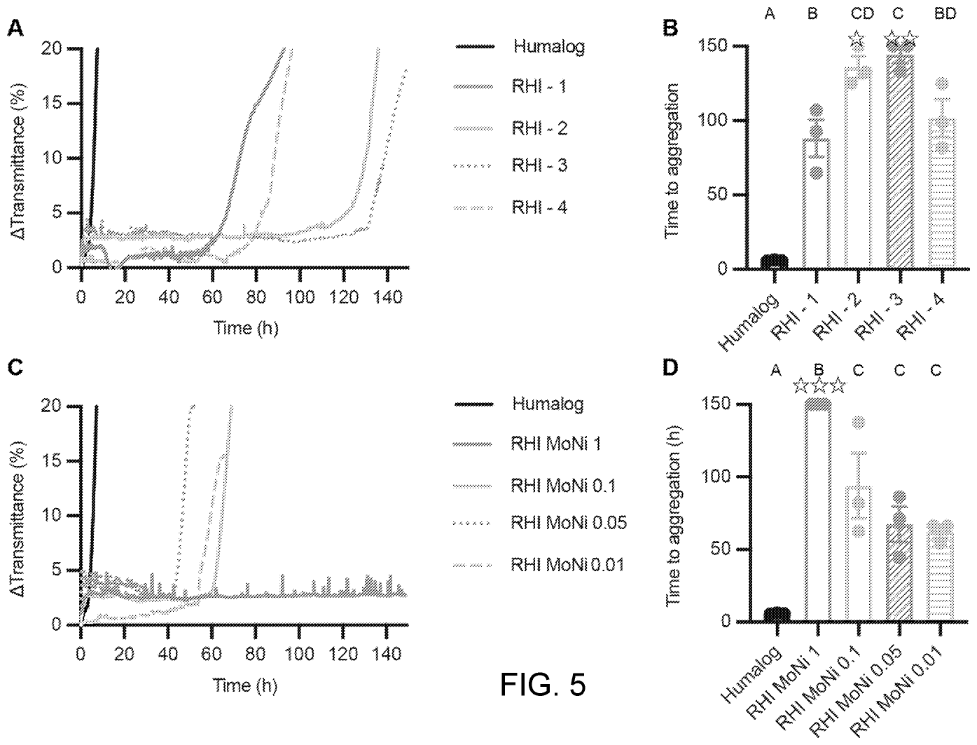

The stability of exemplary regular human insulin (RHI) formulations was assessed. FIG. 5 is a set of graphs showing RHI formulation stability. (Panel A) Change in transmittance traces and (Panel B) Time to aggregation of formulations with different antimicrobial preservatives: RHI—1 (meta-cresol), RHI—2 (meta-cresol+phenoxyethanol), RHI—3 (phenoxyethanol), RHI—4 (methylparaben+propylparaben). (Panel C) Change in transmittance traces and (Panel D) Time to aggregation of phenoxyethanol formulation (RHI—3) with different concentrations of MoNi_{23%} polymer excipient. Comparison of stability by aggregation times (tA), defined as the time to a change in transmittance (λ=540 nm) of 10% or greater following stressed aging (i.e., continuous agitation at 37° C.). Data shown are average transmittance traces for n=3 samples per group and error bars are standard error mean. * represents a sample that did not aggregate before the assay ended at 150 hours. A one-way ANOVA with a Tukey-Kramer correction for multiple comparisons was performed in GraphPad Prism 9, and each formulation was compared to all other formulations. Formulations connected by the same letter label are not significantly different.

Adjusted p values are listed in Tables 9-10, below. α<0.05.

TABLE 9
Adjusted P values for FIG. 5 (Panel B)

| Comparison | Mean difference | Adjusted P Value |
|---|---|---|
| Humalog vs. RHI - 1 | −82.27 | 0.0005 |
| Humalog vs. RHI - 2 | −130.0 | <0.0001 |
| Humalog vs. RHI - 3 | −138.5 | <0.0001 |
| Humalog vs. RHI - 4 | −95.82 | 0.0001 |
| RHI - 1 vs. RHI - 2 | −47.73 | 0.0233 |
| RHI - 1 vs. RHI - 3 | −56.23 | 0.0084 |
| RHI - 1 vs. RHI - 4 | −13.55 | 0.8154 |
| RHI - 2 vs. RHI - 3 | −8.500 | 0.9578 |
| RHI - 2 vs. RHI - 4 | 34.18 | 0.1220 |
| RHI - 3 vs. RHI - 4 | 42.68 | 0.0433 |

TABLE 10
Adjusted P values for FIG. 5 (Panel D)

| | Mean | Adjusted |
|---|---|---|
| Comparison | difference | P Value |
| Humalog vs. RHI MoNi_{23%} 1 | −144.2 | <0.0001 |
| Humalog vs. RHI MoNi_{23%} 0.1 | −88.17 | 0.0008 |
| Humalog vs. RHI MoNi_{23%} 0.05 | −61.55 | 0.0139 |
| Humalog vs. RHI MoNi_{23%} 0.01 | −56.54 | 0.0244 |
| Humalog vs. RHI MoNi_{23%} 0 | −144.2 | <0.0001 |
| RHI MoNi_{23%} 1 vs. RHI MoNi_{23%} 0.1 | 56.00 | 0.0260 |
| RHI MoNi_{23%} 1 vs. RHI MoNi_{23%} 0.05 | 82.62 | 0.0014 |
| RHI MoNi_{23%} 1 vs. RHI MoNi_{23%} 0.01 | 87.63 | 0.0008 |
| RHI MoNi_{23%} 1 vs. RHI MoNi_{23%} 0 | 0.000 | >0.9999 |
| RHI MoNi_{23%} 0.1 vs. RHI MoNi_{23%} 0.05 | 26.62 | 0.5102 |
| RHI MoNi_{23%} 0.1 vs. RHI MoNi_{23%} 0.01 | 31.63 | 0.3405 |
| RHI MoNi_{23%} 0.1 vs. RHI MoNi_{23%} 0 | −56.00 | 0.0260 |
| RHI MoNi_{23%} 0.05 vs. RHI MoNi_{23%} 0.01 | 5.010 | 0.9993 |
| RHI MoNi_{23%} 0.05 vs. RHI MoNi_{23%} 0 | −82.62 | 0.0014 |
| RHI MoNi_{23%} 0.01 vs. RHI MoNi_{23%} 0 | −87.63 | 0.0008 |

### Example 2—Excipients and Insulin Associate State

SEC-MALS research has indicated that phenoxyethanol increases the proportion of monomers in formulation compared to meta-cresol. However, measurements using SEC-MALS require samples to be formulated at very high concentrations (approximately 10-fold formulation concentrations) so that the insulin is at relevant concentrations when it passes the detector. Further, shear forces as insulin travels along the column could result in dissociation of higher order species. Thus, the effects of antimicrobial preservatives on insulin association state were further explored using analytical ultracentrifugation. Analytical ultracentrifugation is a valuable method for examining association states because sedimentation coefficients can be used to identify different oligomers and the relative amounts of each species can be determined using sedimentation velocity (Tables 5, 6).

It was observed that insulin lispro formulations without zinc, regardless of preservative, had fewer hexamers compared to commercial Humalog. Meta-cresol as the only preservative in a zinc-free lispro formulation (lispro-1) resulted in the lower monomer content at 39%. The meta-cresol-phenoxyethanol combination (lispro-2) and phenoxyethanol (lispro-3) resulted in similar insulin lispro association states, both with 57% monomers. The preservative combination of methylparaben and propylparaben (lispro-4) was successful at increasing monomer and dimer content, but had a greater percentage of hexamers (19% hexamers) compared to formulations containing phenoxyethanol (lispro 2 and 3: 0-0.5% hexamers). Glycerol was used as a tonicity agent for all formulations since it is regularly used in commercial formulations and resulted in the highest monomer content when previously tested with SEC-MALS.

In contrast to the lispro formulations, zinc-free regular human insulin resulted in low monomer content for all preservatives. The highest monomer content in zinc-free regular human insulin formulations was seen in the phenoxyethanol formulation (RHI-3), which contained 22% monomers, 8% dimers/trimers, and 70% hexamers. Notably, there was not a higher dimer/trimer content observed in these zinc-free formulations. The primary difference in association state equilibria between rapid-acting insulin analogues and regular human insulin in the absence of zinc is the propensity for dimerization. Regular human insulin has 300-fold higher affinity for dimerization compared to insulin lispro. In contrast, the affinity for hexamer formation is only ˜4-fold greater for regular human insulin. See, e.g., Brems et al., Protein Engineering, Design and Selection 5(6):527-33 (1992). It should be noted that higher ordered structures as reported here are thought to be reversible aggregates such as octamers, rather than high molecular weight polymer or amyloid fibril species.

### Example 3—Excipients and Formulation Stability

The balance between the monomer content of an insulin formulation and stability remains a challenge in developing next-generation insulin formulations. Here, acrylamide copolymer excipient, 4-acryloylmorpholine77%-N-isopropylacrylamide23% (MoNi_{23%}), was used as a stabilizing agent in the zinc-free insulin formulations. The stability of the formulations was evaluated using a stressed aging assay to measure insulin aggregation (FIG. 4, panels A-B). The formulations with the greatest monomer content (lispro-3, RHI-3) were then tested with different concentrations of MoNi_{23%} to assess the optimal polymer concentration required to attain sufficient stability for commercial viability (FIG. 4, panels C-D).

Formulations containing the MoNi_{23%} polymer excipient (0.1 mg/mL polymer) with meta-cresol and/or phenoxyethanol (lispro-1 tA=41±8 h, P=0.0001; lispro-2 tA=42±2 h, P=0.0001; lispro-3 tA=36±4 h, P=0.0001) had extended stability in the stressed aging assay compared to commercial Humalog (tA=5.8±0.4 h) (FIG. 4, panels A-B). Similar times to aggregation were seen between commercial Humalog and the paraben-containing formulation (lispro-4 tA=12±3 h, P=0.2666). Testing the stability of lispro-3 with different concentrations of MoNi_{23%} (FIGS. 4, panels C-D), showed that the stability of formulations with high monomer content was dependent on polymer concentration. Formulations containing 1 mg/mL (140±20 hours; P=0.0001) and 0.1 mg/mL (45±4 hours, P=0.0001) MoNi_{23%} demonstrated extended stability compared to Humalog. It was observed that the order of magnitude increased in MoNi_{23%} concentration aggregation to 20±3 hours, aggregating approximately twice as fast as the formulation with 0.1 mg/mL polymer excipient (P=0.005). However, compared to commercial Humalog the time to aggregation for 0.05 mg/mL (TA: 20±3 hours; p=0.061), 0.01 mg/mL (TA: 7.1±0.3 hours; p=0.999), and 0 mg/mL (TA: 9±2 hours; p=0.965) are not statistically different indicating that lower concentrations of MoNi_{23%} provide limited stability advantages. Since insulin aggregation is a stochastic process, it becomes difficult to differentiate between formulations that have very rapid times to aggregation.

Zinc-free formulations that were prepared with regular human insulin were more stable than zinc-free lispro formulations. This was likely due to reduced monomer content and the greater percentage of stable insulin hexamers in formulation observed in the analytical ultracentrifugation data. All four zinc-free formulations with MoNi_{23%} polymer excipient (0.1 mg/mL polymer) demonstrated extended stability compared to commercial Humalog (RHI-1 tA=90±20 h, P=0.0005; RHI-2 tA=140±13 h, P=0.0001; RHI-3 tA=140±10 h, P=0.0001; RHI-4 tA=100±20 h, P=0.0001; Humalog tA=5.8±0.4 h). No statistical difference was observed between the zinc-free regular human insulin formulations. When different concentrations of MoNi_{23%} were tested in combination with RHI-3, all formulations also had greater stability compared to Humalog.

The results demonstrate that the use of phenoxyethanol as an antimicrobial excipient contributed to high monomer content and eliminated the presence of insulin hexamers in formulation, which are both important characteristics of an ultrafast insulin formulation. These examples also showed that under the tested formulation conditions, zinc-free regular human insulin remained primarily hexameric. The stressed aging assays identified that the stability imbued by amphiphilic copolymer excipient MoNi_{23%} was concentration dependent, and concentrations of 0.1 mg/mL extended the stability of a mostly monomeric zinc-free lispro formulation compared to Humalog. Overall, these results demonstrate how certain excipients modify formulation properties.

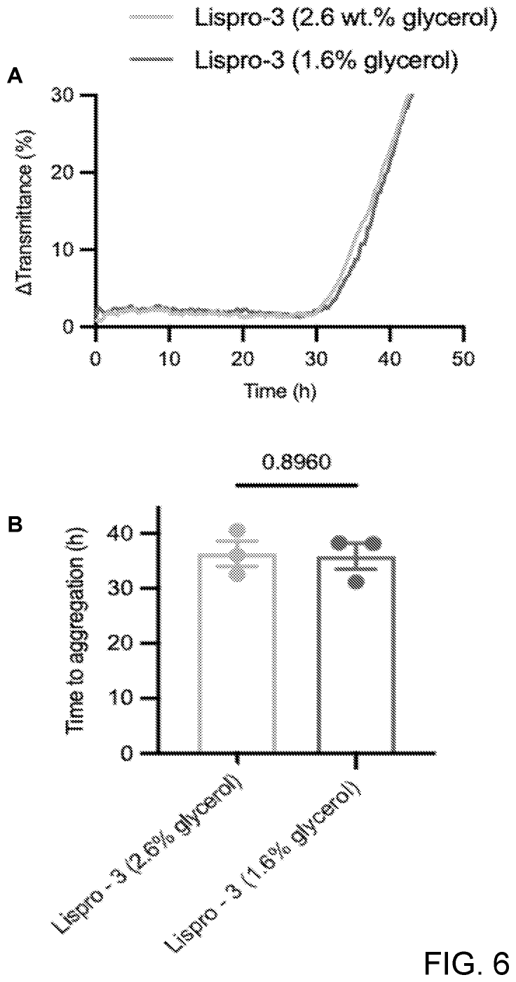

The stability of lispro-3 formulations with different glycerol concentrations was assessed using the methods described above. Here, two variations on the lispro-3 formulation described herein were prepared that differ by the amount of glycerol added (1.6 wt % versus 2.6 wt %). A change in glycerol concentration from 1.6 wt. % as is common in commercial formulations to 2.6 wt. % was shown not to have an effect on formulation stability. FIG. 6, Panel A shows the observed change in transmittance traces for the two exemplary lispro-3 formulations, and FIG. 6, Panel B, shows the time to aggregation. Time to aggregation was compared using a two-tailed t-test (α<0.05) in GraphPad Prism 9.

### Example 4—U400 Insulin Formulation Stability Comparison

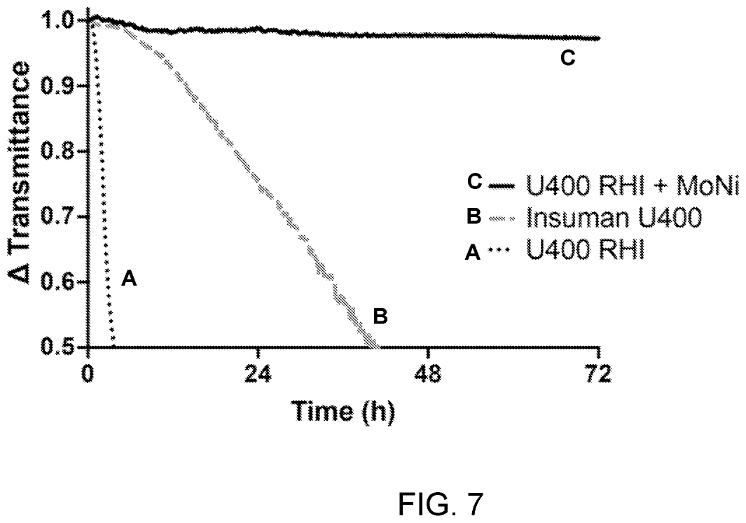

Three formulations with and without MoNi polymer excipient were prepared and evaluated using a stressed aging assay (continuous agitation at 50° C.) to measure insulin aggregation (FIG. 7). The three formulations tested were as follows:
Formulation A (FIG. 7, dotted line A): regular human insulin (RHI) U400, 2.5 mg/mL meta-cresol, 16 mg/mL glycerol, and 10 mM phosphate buffer.
Formulation B (FIG. 7, broken gray line B): a commercial insulin drug product (INSUMAN® U400, Sanofi-Aventis (recombinant human insulin)).
Formulation C (FIG. 7, solid black line C): RHI U400, 2.5 mg/mL meta-cresol, 16 mg/mL glycerol, 10 mM phosphate buffer, and 1 mg/mL MoNi_{23%}.

Formulations A-C were aliquoted 150 μL per well (n=3/group) in a clear 96-well plate and sealed with optically clear and thermally stable seal (VWR). The plate was incubated in a microplate reader (BioTek SynergyH1 microplate reader) at 50° C. with continuous agitation (567 cpm). Absorbance readings were taken every 10 minutes at 540 nm for the duration of the experiment. The formation of insulin aggregates led to light scattering and a reduction in the transmittance of samples (time to aggregation=time to 10% change in transmittance).

As shown in FIG. 7, Formulation C containing the MoNi_{23%} polymer excipient (1 mg/mL polymer) had minimal change in transmittance in the stressed aging assay compared to a commercial insulin drug product (Formulation B, INSUMAN® U400, Sanofi-Aventis (recombinant human insulin)) (FIG. 7, solid black line vs broken gray line); and Formulation A (RHI U400 formulation without MoNi_{23%} excipient) (FIG. 7, solid black line vs dotted line).

The results demonstrate that the use of a MoNi_{23%} polymer excipient (e.g., in Formulation C) contributed to extended stability of the insulin formulation, when compared to a commercial formulation (Formulation B), and an equivalent formulation lacking the MoNi_{23%} excipient (Formulation A).

### EQUIVALENTS AND INCORPORATION BY REFERENCE

While the various embodiments of the present disclosure have been particularly shown and described with reference to a preferred embodiment and various alternate embodiments, it will be understood by persons skilled in the relevant art that various changes in form and details can be made herein without departing from the spirit and scope of the disclosure.

All publications, patents, patent applications, and other documents cited within the body of the instant specification including U.S. Provisional Patent Application No. 63/389,708, and U.S. Provisional Patent Application No. 63/522,786 are hereby incorporated by reference in their entireties for all purposes to the same extent as if each individual publication, patent, patent application, or other document were individually indicated to be incorporated by reference for all purposes.

What is claimed is:

1. A composition comprising:
a polyacrylamide-based copolymer comprising:
a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof; and
a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), N-phenylacrylamide (PHE), and combinations thereof;
a preservative; and
insulin, or an analog thereof.

2. The composition of claim 1, wherein the preservative is selected from meta-cresol, phenoxyethanol, methylparaben, propylparaben, and combinations thereof.

3. The composition of claim 1 or 2, wherein the water-soluble carrier monomer is selected from MORPH, MPAM, and combinations thereof.

4. The composition of claim 3, wherein the water-soluble carrier monomer comprises MORPH.

5. The composition of claim 3, wherein the water-soluble carrier monomer comprises MPAM.

6. The composition of any one of claims 1-5, wherein the functional dopant monomer is selected from AMP, TMA, TBA, PHE, and combinations thereof.

7. The composition of claim 6, wherein the functional dopant monomer comprises AMP.

8. The composition of claim 6, wherein the functional dopant monomer comprises TMA.

9. The composition of claim 6, wherein the functional dopant monomer comprises TBA.

10. The composition of claim 6, wherein the functional dopant monomer comprises PHE.

11. The composition of any one of claims 1-5, wherein the functional dopant monomer is selected from DEA, TRI, PHE, NIP, and combinations thereof.

12. The composition of claim 11, wherein the functional dopant monomer comprises DEA.

13. The composition of claim 11, wherein the functional dopant monomer comprises TRI.

14. The composition of claim 11, wherein the functional dopant monomer comprises PHE.

15. The composition of claim 11, wherein the functional dopant monomer comprises NIP.

16. The composition of claim 1, wherein:
the water-soluble carrier monomer is selected from MPAM, MORPH, and combinations thereof, and
the functional dopant monomer is selected from NIP, PHE, and combinations thereof.

17. The composition of claim 16, wherein:
the water-soluble carrier monomer comprises MPAM; and
the functional dopant monomer comprises NIP.

18. The copolymer of claim 16, wherein:
the water-soluble carrier monomer comprises MPAM; and
the functional dopant monomer comprises PHE.

19. The composition of claim 16, wherein:
the water-soluble carrier monomer comprises MORPH; and
the functional dopant monomer comprises NIP.

20. The composition of claim 16, wherein:
the water-soluble carrier monomer comprises MORPH; and
the functional dopant monomer comprises PHE.

21. The composition of any one of claims 1-20, wherein the copolymer comprises 70 wt % to 98 wt % of the water-soluble carrier monomer.

22. The composition of any one of claims 1-21, wherein the copolymer comprises 2 wt % to 30 wt % of the functional dopant monomer.

23. The composition of claim 22, wherein the copolymer comprises:
70 wt % to 85 wt % of MORPH; and
15 wt % to 30 wt % of NIP.

24. The composition of claim 23, wherein the copolymer comprises:
74 wt % to 80 wt % of MORPH; and
20 wt % to 26 wt % of NIP.

25. The composition of claim 23, wherein the copolymer comprises about 77 wt % of MORPH.

26. The composition of any one of claims 1-25, wherein a degree of polymerization of the copolymer is 10 to 500.

27. The composition of claim 26, wherein a degree of polymerization of the copolymer is 20 to 200.

28. The composition of any one of claims 1-27, wherein a number-average molecular weight of the copolymer is 2,000 g/mol to 10,000 g/mol.

29. The composition of claim 28, wherein a number-average molecular weight of the copolymer is 2,000 g/mol to 6,000 g/mol.

30. The composition of claim 29, wherein a number-average molecular weight of the copolymer is 3,000 g/mol to 5,000 g/mol.

31. The composition of any one of claims 1-30, wherein the composition comprises 0.001 wt % to 1 wt % of the copolymer.

32. The composition of claim 31, wherein the composition comprises 0.005 wt % to 0.5 wt % of the copolymer.

33. The composition of claim 32, wherein the composition comprises 0.01 wt % of the copolymer.

34. The composition of any one of claims 1-33, wherein the composition comprises 0.01 wt % to 2 wt % of the preservative.

35. The composition of claim 34, wherein the composition comprises 0.05 wt % to 1.5 wt % of the preservative.

36. The composition of any one of claims 1-35, wherein the preservative comprises phenoxyethanol.

37. The composition of any one of claims 1-36, wherein the preservative comprises phenoxyethanol and meta-cresol.

38. The composition of any one of claims 1-35, wherein the preservative comprises methylparaben and propylparaben.

39. The composition of claim 36, wherein the composition comprises 0.2 wt % to 1.5 wt % of phenoxyethanol.

40. The composition of claim 39, wherein the composition comprises 0.5 wt % to 1.1 wt % of phenoxyethanol.

41. The composition of claim 40, wherein the composition comprises 0.85 wt % of phenoxyethanol.

42. The composition of any one of claims 1-37, wherein the composition comprises:
0.1 wt % or less of meta-cresol; and
0.3 wt % to 1 wt % of phenoxyethanol.

43. The composition of any one of claims 1-35 and 38, wherein the composition comprises:
0.05 wt % to 0.5 wt % of methylparaben; and
0.001 wt % to 0.1 wt % of propylparaben.

44. The composition of claim 43, wherein the composition comprises:
0.09 wt % to 0.2 wt % of methylparaben; and
0.005 wt % to 0.05 wt % of propylparaben.

45. The composition of claim 43 or 44, wherein the ratio of propylparaben to methylparaben is from 1:8 to 1:10.

46. The composition of claim 45, wherein the ratio of propylparaben to methylparaben is 1:9.

47. The composition of any of claims 1-46, wherein the composition further comprises a tonicity modifier.

48. The composition of claim 47, wherein the composition comprises 0.5 wt % to 5 wt % of the tonicity modifier.

49. The composition of claim 48, wherein the composition comprises 1.5 wt % to 3 wt % of the tonicity modifier.

50. The composition of any one of claims 47-49, wherein the tonicity modifier is glycerol, sodium chloride, or a combination thereof.

51. The composition of claim 50, wherein the composition comprises 1.5 wt % to 3 wt % of glycerol.

52. The composition of claim 51, wherein the composition comprises 2.6 wt % of glycerol.

53. The composition of any one of claims 1-52, wherein the composition is substantially free from meta-cresol, preferably including less than 0.1 wt %, less than 0.05 wt %, less than 0.01 wt %, or less than 0.001 wt % of meta-cresol in the composition.

53. The composition of any one of claims 1-49, wherein the composition is substantially free from zinc, preferably including less than 0.1 wt % of zinc, less than 0.05 wt %, less than 0.01 wt %, or less than 0.001 wt % of zinc in the composition.

54. The composition of any one of claims 1-53, wherein the composition comprises 0.17 wt % to 1.75 wt % of the insulin or the analog thereof.

55. The composition of claim 54, wherein the composition comprises 0.35 wt % of the insulin or the analog thereof.

56. The composition of any one of claims 1-55, wherein the insulin or the analog thereof is selected from porcine insulin, bovine insulin, feline insulin, human insulin, recombinant insulin, insulin lispro, HUMALOG®, insulin glargine, LANTUS®, insulin detemir, LEVEMIR®, ACTRAPID®, modern insulin, NOVORAPID®, VELOSULIN®, HUMULIN® M3, HYPURIN®, INSUMAN®, INSULATARD®, MIXTARD® 30, MIXTARD® 40, MIXTARD® 50, insulin aspart, insulin glulisine, insulin isophane, insulin degludec, insulin icodec, insulin zinc extended, NOVOLIN® R, HUMULIN® R, HUMULIN® R regular U-500, NOVOLIN® N, HUMULIN® N, RELION®, AFREZZA®, HUMULIN® 70/30, NOVOLIN® 70/30, NOVOLOG® 70/30, HUMULIN® 50/50, HUMALOG® mix 75/25, insulin aspart protamine-insulin aspart, insulin lispro protamine-insulin lispro, human insulin NPH-human insulin regular, and insulin degludec-insulin aspart.

57. The composition of any one of claims 1-55, wherein the insulin or the analog thereof is insulin aspart.

58. The composition of any one of claims 1-55, wherein the insulin or the analog thereof is human insulin.

59. The composition of any one of claims 1-55, wherein the insulin or the analog thereof is insulin lispro.

60. The composition of claim 59, wherein the composition comprises 0.17 wt % to 1.75 wt % of insulin lispro.

61. The composition of claim 60, wherein the composition comprises 0.35 wt % of insulin lispro.

62. The composition of any one of claims 1-61, wherein the pH of the composition is 4 to 8.

63. The composition of any one of claims 1-62, wherein the composition is aqueous.

64. The composition of any one of claims 1-63, wherein the composition further comprises a buffer.

65. The composition of claim 64, wherein the buffer comprises phosphate, citrate, acetate, TRIS, histidine, or any combination thereof.

66. The composition of claim 65, wherein the buffer comprises a phosphate.

67. The composition of claim 1, comprising:
0.001 wt % to 1 wt % of a polyacrylamide-based copolymer comprising:
70 wt % to 98 wt % of a water-soluble carrier monomer selected from MPAM, MORPH, DMA, HEAM, and AM; and
2 wt % to 30 wt % of a functional dopant monomer selected from TRI, AMP, TMA, NIP, DEA, TBA, and PHE;
0.01 wt % to 2 wt % of a preservative selected from meta-cresol, phenoxyethanol, methylparaben, and propylparaben; and
0.17 wt % to 0.7 wt % of insulin, or an analog thereof.

68. The composition of claim 67, wherein:
the water-soluble carrier monomer is selected from MPAM, and MORPH; and
the functional dopant monomer is selected from NIP, and PHE.

69. The composition of claim 68, wherein the polyacrylamide-based copolymer comprises:
70 wt % to 85 wt % of MORPH; and
15 wt % to 30 wt % of NIP.

70. The composition of any one of claims 67-69, further comprising 0.5 wt % to 5 wt % of a tonicity modifier.

71. The composition of claim 70, wherein the tonicity modifier is glycerol.

72. The composition of any one of claims 67-71, wherein the insulin or the analog thereof is selected from human insulin, insulin aspart, and insulin lispro.

73. The composition of claim 69, comprising:
0.01 wt % of a polyacrylamide-based copolymer comprising:
about 77 wt % of MORPH; and
about 23 wt % of NIP.

74. The composition of claim 73, comprising:
0.85 wt % of phenoxyethanol;
2.6 wt % of glycerol; and
0.35 wt % of insulin lispro.

75. The composition of claim 73, comprising:
0.64 wt % of phenoxyethanol;
0.064 wt % of meta-cresol;
2.6 wt % of glycerol; and
0.35 wt % of insulin lispro.

76. The composition of claim 1, comprising:
0.15 wt % in total of methylparaben and propylparaben;
2.6 wt % of glycerol; and
0.35 wt % of insulin lispro.

77. The composition of claim 76, comprising:
0.135 wt % methylparaben; and
0.015 wt % of propylparaben.

78. The composition of any one of claims 67-77, wherein the composition is substantially free from zinc, preferably including less than 0.1 wt % of zinc, less than 0.05 wt %, less than 0.01 wt %, or less than 0.001 wt % of zinc in the composition.

79. The composition of any one of claims 1-78, wherein no more than 50 wt % of the insulin or the analog thereof is present in the composition in a hexameric or higher-order association state.

80. The composition of any one of claims 1-79, wherein no more than 25 wt % of the insulin or the analog thereof is present in the composition in a hexameric or higher-order association state.

81. The composition of any one of claims 1-80, wherein substantially none of the insulin or the analog thereof is present in the composition in a hexameric or higher-order association state.

82. The composition of any one of claims 1-78, wherein at least 50 wt % of the insulin or the analog thereof is present in the composition in a monomeric, dimeric, trimeric, or tetrameric association state.

83. The composition of any one of claims 1-78, wherein at least 90 wt % of the insulin or the analog thereof is present in the composition in a monomeric, dimeric, trimeric, or tetrameric association state.

84. The composition of any one of claims 1-78, wherein at least 25 wt % of the insulin or the analog thereof is present in the composition in a monomeric association state.

85. The composition of any one of claims 1-78, wherein at least 50 wt % of the insulin or the analog thereof is present in the composition in a monomeric association state.

86. The composition of any one of claims 1-85, having a peak action of 30 minutes or less.

87. The composition of claim 86, having a peak action of 20 minutes or less.

88. The composition of any one of claims 1-87, having a duration of action of 150 minutes or less.

89. A method of treating an elevated glucose level in a subject in need thereof, comprising administering to the subject a therapeutically effective amount of a composition of any one of claims 1-88, wherein the elevated glucose level is associated with insulin deficiency in the subject.

90. A method of managing a blood glucose level in a subject in need thereof, comprising administering to the subject a therapeutically effective amount of a composition of any of claims 1-88.

91. The method of claim 89 or 90, wherein the composition is administered via an infusion pump or an artificial pancreas closed-loop system.

92. Use of a composition for the manufacture of a medicament for treating an elevated glucose level in a subject in need thereof, wherein the composition is of any one of claims 1-88, and wherein the elevated glucose level is associated with insulin deficiency in the subject.

93. Use of a composition for the treatment of an elevated glucose level in a subject in need thereof, wherein the composition is of any one of claims 1-88, and wherein the elevated glucose level is associated with insulin deficiency in the subject.

94. Use of a composition for the manufacture of a medicament for managing a blood glucose level in a subject in need thereof, wherein the composition is of any one of claims 1-88.

95. Use of a composition for the management of a blood glucose level in a subject in need thereof, wherein the composition is of any one of claims 1-88.
